(19) United States
(12) Patent Application Publication
(10) Pub. No.: US 2011/0136924 A1
(43) Pub. Date: Jun. 9, 2011

(54) CATALYST AND PROCESS FOR PRODUCING LIQUEFIED PETROLEUM GAS

(75) Inventors: Kaoru Fujimoto, Fukuoka (JP); Xiaohong Li, Fukuoka (JP); Wenliang Zhu, Tokyo (JP); Qingjie Ge, Tokyo (JP)

(73) Assignee: JAPAN GAS SYNTHESIZE, LTD., Tokyo (JP)

(21) Appl. No.: 12/918,655

(22) PCT Filed: Feb. 20, 2009
(86) PCT No.: PCT/JP2009/053043

§ 371 Date: Feb. 22, 2011

(30) Foreign Application Priority Data
Feb. 20, 2008 (JP) 2008-039296

(51) Int. Cl. C07C 1/00; B01J 29/06

(52) U.S. Cl. 518/700; 502/87

(57) ABSTRACT

The present invention relates to a catalyst for producing a liquefied petroleum gas, which is used for producing a liquefied petroleum gas containing propane or butane as a main component by reacting carbon monoxide and hydrogen, and comprises a Cu—Zn-based methanol synthesis catalyst and a Cu-supported β-zeolite in which at least Cu is supported on a β-zeolite.

### TECHNICAL FIELD

The present invention relates to a catalyst for producing a liquefied petroleum gas containing propane or butane as a main component by reacting carbon monoxide with hydrogen.

The present invention also relates to a process for producing a liquefied petroleum gas containing propane or butane as a main component from a synthesis gas, using the catalyst. The present invention also relates to a process for producing a liquefied petroleum gas containing propane or butane as a main component from a carbon-containing starting material such as a natural gas, using the catalyst.

### BACKGROUND ART

Liquefied petroleum gas (LPG) is a liquefied petroleum-based or natural-gas-based hydrocarbon which is gaseous at an ambient temperature under an atmospheric pressure by compression while optionally cooling. The main component of LPG is propane or butane. LPG is advantageously transportable because it can be stored or transported in a liquid form. Thus, in contrast with a natural gas that requires a pipeline for supply, it has a characteristic that it can be filled in a container to be supplied to any place. For that reason, LPG comprising propane as a main component, i.e. propane gas, has been widely used as a fuel for household and business use. At present, propane gas is supplied to about 25 million households (more than 50% of the total households) in Japan. In addition to household and business use, LPG is used as a fuel for a portable product such as a portable gas burner and a disposable lighter (mainly, butane gas), an industrial fuel and an automobile fuel.

Conventionally, LPG has been produced by 1) collection from a wet natural gas, 2) collection from a stabilization (vapor-pressure regulating) process of crude petroleum, 3) separation and extraction of a product in, for example, a petroleum refining process, or the like.

LPG, particularly propane gas which is used as a household/business fuel, is expected to be in great demand in the future. Thus, it may be very useful to establish an industrially practicable and new process for producing LPG.

As a process for producing LPG, Patent document 1 discloses that a synthesis gas consisting of hydrogen and carbon monoxide is reacted in the presence of a mixed catalyst obtained by physically mixing a methanol synthesis catalyst such as a Cu—Zn-based catalyst, a Cr—Zn-based catalyst and a Pd-based catalyst, specifically a CuO—ZnO—Al₂O₃ catalyst, a Pd/SiO₂ catalyst or a Cr—Zn-based catalyst with a methanol conversion catalyst composed of a zeolite having an average pore size of about 10 Å (1 nm) or more, specifically a Y-type zeolite, to provide a liquefied petroleum gas or a mixture of hydrocarbons similar in composition to LPG. The catalyst described in Patent document 1, however, does not necessarily have sufficient performance.

As a process for producing LPG, Non-patent document 1 discloses that a hybrid catalyst consisting of a methanol synthesis catalyst such as a 4 wt % Pd/SiO₂, a Cu—Zn—Al mixed oxide {Cu:Zn:Al=40:23:37 (atomic ratio)} or a Cu-based low-pressure methanol synthesis catalyst (Trade name: BASF S3-85) and a high-silica Y-type zeolite with SiO₂/Al₂O₃=7.6, which has been subjected to treatment with steam at 450° C. for 1 hour, can be used to produce C2 to C4 paraffins in a selectivity of 69 to 85% via methanol and dimethyl ether from a synthesis gas. However, the catalyst described in Non-patent document 1 may not have sufficient performance, as well as the catalyst described in the above-mentioned Patent document 1.

As a catalyst for producing a liquefied petroleum gas which has excellent catalyst performance, longer catalyst life, and reduced deterioration over time, Patent document 2 discloses a catalyst comprising a Pd-based methanol synthesis catalyst (catalyst in which 0.1 to 10 wt % of Pd is supported on a silica) and a β-zeolite. Patent document 3 discloses a catalyst comprising a catalyst in which an olefin-hydrogenation catalyst component is supported on a Zn—Cr-based methanol synthesis catalyst (catalyst in which 0.005 to 5 wt % of Pd is supported on a Zn—Cr-based methanol synthesis catalyst) and a β-zeolite. However, Pd used in these catalysts for producing a liquefied petroleum gas is very high-priced. Therefore, these catalysts may be unfavorable in terms of cost.

In addition, Non-patent document 2 discloses that a hybrid catalyst consisting of Pd—SiO₂ or Pd, Ca—SiO₂ as a methanol synthesis catalyst, and a β-zeolite or a USY-type zeolite can be used to produce LPG from a synthesis gas. In the catalyst described in Non-patent document 2, however, the amount of Pd in Pd—SiO₂ or Pd, Ca—SiO₂ is 4 wt %, and high-priced Pd is used in relatively large quantities. Therefore, this catalyst may be also unfavorable in terms of cost.

Patent document 4 discloses a catalyst comprising a Cu—Zn-based methanol synthesis catalyst component and a β-zeolite catalyst component in which preferably 0.1 to 1 wt % of Pd is supported on a β-zeolite, which is described as a catalyst for producing a liquefied petroleum gas which is suppressed in deterioration over time and capable of serving as a catalyst in a reaction for producing a liquefied petroleum gas from carbon monoxide and hydrogen under relatively low temperature and pressure conditions. With regard to the durability of the catalyst, Patent document 4 discloses that the catalyst exhibits good activity (e.g., C3+C4 selectivity) for about 300 hours (Example 2). However, in this Example, the temperature is controlled such that the CO conversion is maintained at approximately 80% (the reaction temperature is increased from 270° C. to 295° C. in stepwise). Such a temperature control may be troublesome in a practical process for producing LPG. In addition, this catalyst comprises high-priced Pd. Therefore, this catalyst may be also unfavorable in terms of cost.

### LIST OF REFERENCES

Patent document 1: JP-A-S61-23688
Patent Document 2: JP-A-2006-21100
Patent Document 3: WO 2006/016444 A1
Patent Document 4: WO 2007/094457 A1
Non-patent document 1: “Selective Synthesis of LPG from Synthesis Gas”, Kaoru Fujimoto et al., Bull. Chem. Soc. Jpn., 58, p. 3059-3060 (1985)
Non-patent document 2: “Synthesis of LPG from Synthesis Gas with Hybrid Catalyst”, Qianwen Zhang et al., Dai 33 Kai Sekiyu Sekiyu Kagaku Toronkai Koen Yoshi (the summaries of the 33th Petroleum and Petrochemistry Discussion), p. 179-180, Nov. 17, 2003

### DISCLOSURE OF THE INVENTION

### Problems to be Solved by the Invention

An objective of the present invention is to provide a low-cost catalyst for producing a liquefied petroleum gas, which enables the production of a hydrocarbon containing propane or butane as a main component, i.e. liquefied petroleum gas (LPG), with high activity, high selectivity and high yield by reacting carbon monoxide and hydrogen, and has a longer catalyst life with less deterioration over time.

Another objective of the present invention is to provide a process for stably producing LPG, which has a high concentration of propane and/or butane, from a synthesis gas with high yield for a long period, using the catalyst. A further objective of the present invention is to provide a process for stably producing LPG, which has a high concentration of propane and/or butane, from a carbon-containing starting material such as a natural gas with high yield for a long period.

### Means for Solving the Problems

The present invention relates to the followings.

[1] A catalyst for producing a liquefied petroleum gas, which is used for producing a liquefied petroleum gas containing propane or butane as a main component by reacting carbon monoxide and hydrogen, comprising

a Cu—Zn-based methanol synthesis catalyst; and

a Cu-supported β-zeolite in which at least Cu is supported on a β-zeolite.

[2] The catalyst for producing a liquefied petroleum gas as described in [1], wherein a ratio (by weight) of the Cu—Zn-based methanol synthesis catalyst to the Cu-supported β-zeolite [(Cu—Zn-based methanol synthesis catalyst)/(Cu-supported β-zeolite)] is 0.1 to 5.

[3] The catalyst for producing a liquefied petroleum gas as described in any one of [1] to [2], wherein the Cu—Zn-based methanol synthesis catalyst is a composite oxide containing copper oxide and zinc oxide as a main component, or a composite oxide containing copper oxide and zinc oxide as a main component and having at least one metal supported thereon.

[4] The catalyst for producing a liquefied petroleum gas as described in [3], wherein the composite oxide contains copper oxide and zinc oxide and optionally aluminum oxide and/or chromium oxide in the ratio (by weight) of (copper oxide):(zinc oxide):(aluminum oxide):(chromium oxide)=100:(10 to 70):(0 to 60):(0 to 50).

[5] The catalyst for producing a liquefied petroleum gas as described in any one of [3] to [4], wherein the Cu—Zn-based methanol synthesis catalyst has Zr supported on the composite oxide.

[6] The catalyst for producing a liquefied petroleum gas as described in [5], wherein the amount of Zr supported in the Cu—Zn-based methanol synthesis catalyst is 0.5 wt % to 8 wt %.

[7] The catalyst for producing a liquefied petroleum gas as described in any one of [1] to [6], wherein the β-zeolite as a support for the Cu-supported β-zeolite has a SiO₂/Al₂O₃ ratio of 10 to 150.

[8] The catalyst for producing a liquefied petroleum gas as described in any one of [1] to [7], wherein the amount of Cu supported in the Cu-supported β-zeolite is 0.1 wt % to 15 wt %.

[9] The catalyst for producing a liquefied petroleum gas as described in any one of [1] to [8], wherein the Cu-supported β-zeolite has Cu and Zr supported on the β-zeolite.

[10] The catalyst for producing a liquefied petroleum gas as described in [9], wherein the amount of Zr supported in the Cu-supported β-zeolite is 0.1 wt % to 5 wt %.

[11] The catalyst for producing a liquefied petroleum gas as described in any one of [1] to [10], wherein the Cu—Zn-based methanol synthesis catalyst is a composite oxide containing copper oxide and zinc oxide as a main component on which Zr is supported in an amount of 0.5 wt % to 8 wt %; and the Cu-supported β-zeolite is a β-zeolite with a SiO₂/Al₂O₃ ratio of 10 to 150 on which Cu is supported in an amount of 0.1 wt % to 15 wt % and Zr is supported in an amount of 0.1 wt % to 5 wt %.

[12] A process for producing a liquefied petroleum gas, comprising a step of:

reacting carbon monoxide and hydrogen in the presence of the catalyst as described in any one of [1] to [11], whereby producing a liquefied petroleum gas containing propane or butane as a main component.

[13] The process for producing a liquefied petroleum gas as described in [12], wherein carbon monoxide and hydrogen are reacted at a reaction temperature of 260° C. to 325° C.; a reaction pressure of 1.6 MPa to 4.5 MPa; and a contact time between a starting gas, which contains carbon monoxide and hydrogen, and the catalyst [W/F; ratio of the weight of the catalyst (W; g) to the total flow rate of the starting gas (F; mol/h)] of 2 g·h/mol to 20 g·h/mol.

[14] A process for producing a liquefied petroleum gas, comprising a step of feeding a synthesis gas to a catalyst layer comprising the catalyst as described in any one of [1] to [11], whereby producing a liquefied petroleum gas containing propane or butane as a main component.

[15] A process for producing a liquefied petroleum gas, comprising:

(1) a step of producing a synthesis gas from a carbon-containing starting material and at least one selected from the group consisting of H₂O, O₂ and CO₂; and

(2) a step of producing a liquefied petroleum gas wherein the synthesis gas is fed to a catalyst layer comprising the catalyst as described in any one of [1] to [11], whereby producing a liquefied petroleum gas containing propane or butane as a main component.

The term “synthesis gas” as used herein is a mixed gas comprising hydrogen and carbon monoxide, and is not limited to a mixed gas consisting of hydrogen and carbon monoxide. A synthesis gas may be, for example, a mixed gas comprising carbon dioxide, water, methane, ethane, ethylene and so on. A synthesis gas produced by reforming a natural gas generally contains, in addition to hydrogen and carbon monoxide, carbon dioxide and water vapor. A synthesis gas may be a coal gas produced by coal gasification, or a water gas produced from a coal coke.

### Effect of the Invention

When carbon monoxide and hydrogen are reacted in the presence of a catalyst comprising a methanol synthesis catalyst component and a zeolite catalyst component, the reaction represented by the following formula (I) may proceed to form a hydrocarbon containing propane or butane as a main component, i.e. liquefied petroleum gas (LPG).

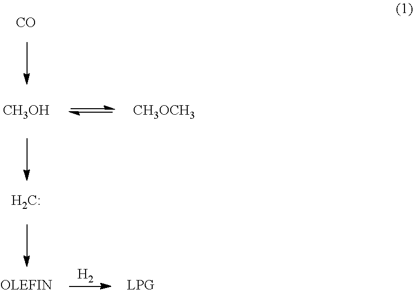

First, on the methanol synthesis catalyst component, methanol is formed from carbon monoxide and hydrogen. Simultaneously, dimethyl ether is also formed by dehydro-dimerization of methanol. Then, methanol thus formed is converted to a lower-olefin hydrocarbon comprising propylene or butene as a main component at an active site in a pore in the zeolite catalyst component. In the reaction, methanol would be dehydrated to form a carbene (H₂C:), which is subjected to polymerization to form a lower olefin. The lower olefin thus formed is released from the pore in the zeolite catalyst component and rapidly hydrogenated on the methanol synthesis catalyst component, to form a paraffin comprising propane or butane as a main component, i.e. LPG.

The term “methanol synthesis catalyst component” as used herein refers to a compound which can act as a catalyst in the reaction of CO+2H₂→CH₃OH. The term “zeolite catalyst component” as used herein refers to a zeolite which can act as a catalyst in a condensation reaction of methanol into a hydrocarbon and/or a condensation reaction of dimethyl ether into a hydrocarbon. Additionally, the methanol synthesis catalyst component is required to act as a catalyst in a hydrogenation reaction of an olefin into a paraffin.

According to the present invention, a methanol synthesis catalyst component to be used is a Cu—Zn-based methanol synthesis catalyst, and a zeolite catalyst component to be used is a Cu-supported β-zeolite in which Cu is supported on a β-zeolite. When combining a Cu—Zn-based methanol synthesis catalyst with a Cu-supported β-zeolite, LPG (propane, butane) may be produced at a temperature of from 260° C. to 325° C., preferably at a temperature equal to or lower than 300° C., more preferably at a temperature equal to or lower than 290° C., with high activity, high selectivity and high yield, which is equal to or higher than a conventional catalyst. In addition, the catalyst for producing a liquefied petroleum gas according to the present invention has reduced deterioration over time, and a longer catalyst life. The catalyst of the present invention has much higher stability and durability, as compared with a conventional catalyst.

Accordingly, when using the catalyst for producing a liquefied petroleum gas according to the present invention, propane and/or butane, i.e. LPG, may be produced with high activity and high yield for a longer period.

Moreover, any conventional catalyst for producing a liquefied petroleum gas which has excellent performance comprises high-priced Pd. In contrast, the catalyst of the present invention does not comprise Pd. Thus, the catalyst of the present invention is more inexpensive than conventional catalysts.

A Cu-supported β-zeolite to be used in the present invention may have a metal other than Cu, for example, Zr, which is supported on the zeolite. A Cu—Zn-based methanol synthesis catalyst to be used in the present invention may be selected, without limitation, from those which contains Cu and Zn, and can act as a catalyst in the reaction: CO+2H₂CH₃OH. The common Cu—Zn-based methanol synthesis catalysts are a composite oxide composed mainly of copper oxide and zinc oxide (Cu—Zn composite oxide), and a composite oxide composed mainly of copper oxide and zinc oxide and containing aluminum oxide and/or chromium oxide and/or other metal oxides as an additive component (for example, Cu—Zn—Al composite oxide and Cu—Zn—Cr composite oxide). According to the present invention, catalysts in which a metal such as Zr is supported on these common Cu—Zn-based methanol synthesis catalysts, as well as common Cu—Zn-based methanol synthesis catalysts, may be suitably used.

According to the present invention, a preferable catalyst may be the combination of a methanol synthesis catalyst component in which Zr is supported in an amount of 0.5 wt % to 8 wt % on a Cu—Zn-based methanol synthesis catalyst such as Cu—Zn composite oxide, Cu—Zn—Al composite oxide and Cu—Zn—Cr composite oxide; and a zeolite catalyst component in which Cu is supported in an amount of 0.1 wt % to 15 wt % and Zr is supported in an amount of 0.1 wt % to 5 wt % on a β-zeolite, preferably a β-zeolite with a SiO₂/Al₂O₃ ratio of 10 to 150, because the stability of the catalyst may be greatly improved, while maintaining high activity and high selectivity.

The reaction conditions are also important for stably producing LPG for a long period with high conversion, high selectivity and high yield. The present invention may be particularly effective when carbon monoxide and hydrogen are reacted in the presence of the catalyst of the present invention at a reaction temperature of 260° C. to 325° C.; a reaction pressure of 1.6 MPa to 4.5 MPa; and W/F of 7 g·h/mol to 20 g·h/mol.

### BRIEF DESCRIPTION OF THE DRAWINGS

FIG. 1 is a process flow diagram showing a main configuration in an example of an LPG producing apparatus suitable for conducting the process for LPG production according to the present invention.

FIG. 2 is a graph showing the results of the LPG synthesis reactions at various reaction temperatures using a (Cu—Zn+0.5% Cu-β-37) catalyst in Example 1.

FIG. 3 is a graph showing the results of the LPG synthesis reactions at various reaction temperatures using a (Cu—Zn+0.5% Cu-β-350) catalyst in Example 2.

FIG. 4 is a graph showing the results of the LPG synthesis reactions at various reaction temperatures using a (Cu—Zn+5.0% Cu/β-37) catalyst in Example 3.

FIG. 5 is a graph showing the results of the LPG synthesis reactions at various reaction temperatures using a (Cu—Zn+10% Cu/β-37) catalyst in Example 4.

FIG. 6 is a graph showing the results of the LPG synthesis reactions at various reaction pressures using a (Cu—Zn+5.0% Cu/β-37) catalyst in Example 5.

FIG. 7 is a graph showing the results of the LPG synthesis reactions at various W/Fs using a (Cu—Zn+5.0% Cu/β-37) catalyst in Example 6.

FIG. 8 is a graph showing the result (change in the conversion of carbon monoxide into hydrocarbon (CH), the conversion of carbon monoxide into carbon dioxide by shift reaction, and the composition of the synthesized hydrocarbon over time) of the LPG synthesis reaction using a (Cu—Zn+5.0% Cu/β-37) catalyst in Example 7.

FIG. 9 is a graph showing the result (change in the conversion of carbon monoxide into hydrocarbon (CH), the conversion of carbon monoxide into carbon dioxide by shift reaction, and the composition of the synthesized hydrocarbon over time) of the LPG synthesis reaction using a (Cu—Zn+10% Cu/β-37) catalyst in Example 8.

FIG. 10 is a graph showing the result (change in the conversion of carbon monoxide into hydrocarbon (CH), the conversion of carbon monoxide into carbon dioxide by shift reaction, and the composition of the synthesized hydrocarbon over time) of the LPG synthesis reaction using a (Cu—Zn+2.0% Cu/β-37) catalyst in Example 9.

FIG. 11 is a graph showing the result (change in the conversion of carbon monoxide into hydrocarbon (CH), the conversion of carbon monoxide into carbon dioxide by shift reaction, and the composition of the synthesized hydrocarbon over time) of the LPG synthesis reaction using a [Cu—Zn+(5.0% Cu+2.5% Zn)/β-371 catalyst in Example 10.

FIG. 12 is a graph showing the result (change in the conversion of carbon monoxide into hydrocarbon (CH), the conversion of carbon monoxide into carbon dioxide by shift reaction, and the composition of the synthesized hydrocarbon over time) of the LPG synthesis reaction using a [Cu—Zn+(5.0% Cu+2.5% Zr)/β-37] catalyst in Example 11.

FIG. 13 is a graph showing the result (change in the conversion of carbon monoxide into hydrocarbon (CH), the conversion of carbon monoxide into carbon dioxide by shift reaction, and the composition of the synthesized hydrocarbon over time) of the LPG synthesis reaction using a [(Cu—Zn+2.5% Cr)+(5.0% Cu+2.5% Zr)/β-37] catalyst in Example 12.

FIG. 14 is a graph showing the result (change in the conversion of carbon monoxide into hydrocarbon (CH), the conversion of carbon monoxide into carbon dioxide by shift reaction, and the composition of the synthesized hydrocarbon over time) of the LPG synthesis reaction using a [(Cu—Zn+2.5% Zr)+(5.0% Cu+2.5% Zr)/β-37] catalyst in Example 13.

### DESCRIPTION OF THE MAIN SYMBOLS

1: a reformer
1a: a reforming catalyst layer
2: a reactor
2a: a catalyst layer
3, 4, 5: lines.

### BEST MODE FOR CARRYING OUT THE INVENTION

### 1. Catalyst for Producing a Liquefied Petroleum Gas According to the Present Invention

A catalyst for producing a liquefied petroleum gas according to the present invention comprises at least one Cu—Zn-based methanol synthesis catalyst, and at least one Cu-supported β-zeolite in which at least Cu is supported on a β-zeolite.

A catalyst for producing a liquefied petroleum gas of the present invention may comprise other additive components as long as its intended effect would not be impaired.

A ratio of the Cu—Zn-based methanol synthesis catalyst to the Cu-supported β-zeolite (Cu—Zn-based methanol synthesis catalyst/Cu-supported β-zeolite; by weight) is preferably 0.1 or more, more preferably 0.5 or more. A ratio of the Cu—Zn-based methanol synthesis catalyst to the Cu-supported β-zeolite (Cu—Zn-based methanol synthesis catalyst/Cu-supported β-zeolite; by weight) is preferably 5 or less, more preferably 3 or less. When a ratio of the Cu—Zn-based methanol synthesis catalyst to the Cu-supported β-zeolite is controlled to within the above range, propane and/or butane may be produced with higher selectivity and higher yield.

A Cu—Zn-based methanol synthesis catalyst as a methanol synthesis catalyst component acts as a methanol synthesis catalyst and a hydrogenation catalyst for an olefin. A Cu-supported β-zeolite as a zeolite catalyst component acts as a solid acid zeolite catalyst, whose acidity is adjusted, in a condensation reaction of methanol and/or of dimethyl ether into hydrocarbon. A ratio of the methanol synthesis catalyst component to the zeolite catalyst component is, therefore, reflected in a relative ratio of the ability to form methanol and the ability to hydrogenate an olefin to the ability to form a hydrocarbon from methanol in the catalyst of the present invention has. In the present invention, when reacting carbon monoxide and hydrogen to produce a liquefied petroleum gas comprising propane or butane as a main component, carbon monoxide and hydrogen must be sufficiently converted into methanol by the action of a methanol synthesis catalyst component, and methanol produced must be sufficiently converted, by the action of a zeolite catalyst component, into an olefin comprising propylene or butene as a main component, which must be converted into a liquefied petroleum gas comprising propane or butane as a main component by the action of a methanol synthesis catalyst component.

When a ratio of the Cu—Zn-based methanol synthesis catalyst to the Cu-supported β-zeolite (Cu—Zn-based methanol synthesis catalyst/Cu-supported β-zeolite; by weight) is 0.1 or more, more preferably 0.5 or more, carbon monoxide and hydrogen may be converted into methanol with higher conversion. In addition, when a ratio of the Cu—Zn-based methanol synthesis catalyst to the Cu-supported β-zeolite (Cu—Zn-based methanol synthesis catalyst/Cu-supported β-zeolite; by weight) is 0.8 or more, methanol produced may be converted into a liquefied petroleum gas comprising propane or butane as a main component with higher selectivity.

On the other hand, when a ratio of the Cu—Zn-based methanol synthesis catalyst to the Cu-supported β-zeolite (Cu—Zn-based methanol synthesis catalyst/Cu-supported β-zeolite; by weight) is 5 or less, more preferably 3 or less, methanol produced may be converted into a liquefied petroleum gas comprising propane or butane as a main component with higher conversion.

A ratio of the Cu—Zn-based methanol synthesis catalyst to the Cu-supported β-zeolite is not limited to the above range, and may be appropriately determined, depending on the types of a methanol synthesis catalyst component and a zeolite catalyst component, and the like.

(Methanol Synthesis Catalyst Component; Cu—Zn-Based Methanol Synthesis Catalyst)

A methanol synthesis catalyst component to be used in the present invention is a Cu—Zn-based methanol synthesis catalyst,

A Cu—Zn-based methanol synthesis catalyst to be used may be selected, without limitation, from those which contains Cu and Zn, and can act as a catalyst in the reaction: CO+2H₂→CH₃OH, and any known Cu—Zn-based methanol synthesis catalyst may be used. A commercially available Cu—Zn-based methanol synthesis catalyst may be also used.

A commonly-used Cu—Zn-based methanol synthesis catalyst is a composite oxide composed mainly of copper oxide and zinc oxide (Cu—Zn composite oxide), and a composite oxide composed mainly of copper oxide and zinc oxide and containing aluminum oxide and/or chromium oxide and/or other metal oxides as an additive component (for example, Cu—Zn—Al composite oxide and Cu—Zn—Cr composite oxide).

A Cu—Zn-based methanol synthesis catalyst to be used in the present invention may preferably contain copper oxide and zinc oxide and optionally aluminum oxide and chromium oxide in the ratio (by weight) of (copper oxide):(zinc oxide):(aluminum oxide):(chromium oxide)=100:(10 to 70):(0 to 60):(0 to 50), more preferably (copper oxide)=(zinc oxide) (aluminum oxide):(chromium oxide)=100:(20 to 60):(0 to 40):(0 to 40). When using a Cu—Zn-based methanol synthesis catalyst having a ratio of oxides within the above range, higher catalytic activity may be achieved, and propane and/or butane may be produced with higher conversion, higher selectivity and higher yield.

According to the present invention, not only commonly-used Cu—Zn-based methanol synthesis catalysts as described above but also catalysts in which at least one metal is supported on these commonly-used Cu—Zn-based methanol synthesis catalysts may be used. The metal is preferably supported on a Cu—Zn-based methanol synthesis catalyst (a composite oxide composed mainly of copper oxide and zinc oxide) in a highly dispersed manner.

A particularly preferable metal-supported Cu—Zn-based methanol synthesis catalyst may be a catalyst in which Zr is supported on a Cu—Zn-based methanol synthesis catalyst as described above (a composite oxide composed mainly of copper oxide and zinc oxide, which may optionally contain aluminum oxide, chromium oxide and so on). When using a Zr-supported Cu—Zn-based methanol synthesis catalyst in combination with a Cu-supported β-zeolite, particularly a Cu, Zr-supported β-zeolite in which Cu and Zr are supported on a β-zeolite, the stability and durability of the catalyst may be greatly improved.

A supported metal such as Zr may not be necessarily contained as a metal, and may be contained in the form of an oxide, a nitrate, a chloride and the like. In such a case, prior to the reaction, the catalyst may be subjected to reduction by hydrogen, for example, to convert the supported metal into a metal or a metal oxide, if necessary.

The amount of the supported Zr in the Cu—Zn-based methanol synthesis catalyst is preferably 0.5 wt % or more, more preferably 1 wt % or more, particularly preferably 1.5 wt % or more. In addition, the amount of the supported Zr in the Cu—Zn-based methanol synthesis catalyst is preferably 8 wt % or less, more preferably 5 wt % or less, in the light of dispersibility and economical efficiency.

The methanol synthesis catalyst component as described above (Zr-supported Cu—Zn-based methanol synthesis catalyst) may be a Cu—Zn-based methanol synthesis catalyst on which another component, in addition to Zr, is supported as long as the desired effects of the catalyst are maintained.

The Cu—Zn-based methanol synthesis catalyst (including metal-supported Cu—Zn-based methanol synthesis catalyst) may be used alone or in combination of two or more.

A Cu—Zn-based methanol synthesis catalyst such as Cu—Zn composite oxide, Cu—Zn—Al composite oxide and Cu—Zn—Cr composite oxide may be prepared by a known method such as a precipitation method. A metal-supported Cu—Zn-based methanol synthesis catalyst such as Zr-supported Cu—Zn-based methanol synthesis catalyst may be prepared by carrying a metal such as Zr or a metal compound onto a Cu—Zn-based methanol synthesis catalyst, which may be prepared by a precipitation method and the like, or commercially available, by a known method such as an impregnation method.

(Zeolite Catalyst Component; Cu-Supported β-Zeolite)

A zeolite catalyst component to be used in the present invention is a Cu-supported fβ-zeolite in which at least Cu is supported on a β-zeolite.

Cu may not be necessarily contained as a metal, and may be contained in the form of an oxide, a nitrate, a chloride and the like. In such a case, in order to achieve higher catalytic activity, the catalyst may be preferably subjected to reduction by hydrogen, for example, to convert Cu into metallic copper, prior to the reaction. The reduction treatment condition to activate Cu may be appropriately determined.

In addition, Cu is preferably supported on a β-zeolite in a highly dispersed manner.

The amount of the supported Cu in the Cu-supported β-zeolite is preferably 0.1 wt % or more, more preferably 1 wt % or more, particularly preferably 3 wt % or more. In addition, the amount of the supported Cu in the Cu-supported β-zeolite is preferably 15 wt % or less, more preferably 10 wt % or less, particularly preferably 8 wt % or less. When the amount of the supported Cu in the Cu-supported β-zeolite is within the above range, propane and/or butane may be stably produced with higher conversion, higher selectivity and higher yield for a longer period. A substantial amount of Cu must be supported on a β-zeolite to achieve the remarkable effect of the present invention. However, when the amount of the supported Cu in the Cu-supported β-zeolite is excessively high, the catalytic activity may rapidly decrease.

The β-zeolite as a support for the Cu-supported β-zeolite is preferably, but not limited to, a β-zeolite with a SiO₂/Al₂O₃ ratio of 10 to 150. When using a β-zeolite with a SiO₂/Al₂O₃ ratio of 10 to 150, higher catalytic activity and higher selectivity for propane and butane may be achieved. The SiO₂/Al₂O₃ ratio of β-zeolite is more preferably 100 or less, particularly preferably 50 or less. In addition, the SiO₂/Al₂O₃ ratio of β-zeolite is more preferably 20 or more, particularly preferably 30 or more.

The β-zeolite may contain an element other than Si and Al in the lattice.

The Cu-supported β-zeolite to be used in the present invention may have at least one other metal together with Cu, which is supported on the β-zeolite. When a certain metal, together with Cu, is supported on a β-zeolite, Cu may be more stably supported on the fβ-zeolite.

Specific examples of the supported metal or metal compound may include Zr, Zn, Cr, Ni, Mo and Co. In the light of cost, it is not preferred that a noble metal including Pd is supported on the β-zeolite, as described above, although a small amount of Pd in addition to Cu may be supported on the β-zeolite.

A particularly preferable supported metal may be Zr. When Zr in addition to Cu is supported on a β-zeolite, the stability and durability of the catalyst may be further improved, while maintaining high activity and high selectivity for propane and butane.

In the case where Cu and Zr are supported on a β-zeolite, the amount of the supported Zr is preferably 0.1 wt % or more, more preferably 1 wt % or more, particularly preferably 2 wt % or more. When the amount of the supported Zr is within the above range, the stability and durability of the catalyst may be sufficiently improved.

In addition, the amount of the supported Zr is preferably 5 wt % or less, more preferably 3 wt % or less. An excessively large amount of the supported Zr may lead to lower activity and lower selectivity for LPG.

A particularly preferable Cu-supported β-zeolite to be used in the present invention may be a β-zeolite with a SiO₂/Al₂O₃ ratio of 10 to 150, more preferably 10 to 50, on which Cu is supported in an amount of 0.1 wt % to 15 wt % and Zr is supported in an amount of 0.1 wt % to 5 wt %.

According to the present invention, the Cu-supported β-zeolite may be a β-zeolite on which another component, in addition to Cu, or Cu and Zr, is supported as long as the desired effects of the catalyst are maintained.

The Cu-supported β-zeolite (including Cu-supported β-zeolite in which a metal other than Cu is supported on a β-zeolite) may be used alone or in combination of two or more.

A Cu-supported β-zeolite in which Cu and optionally Zr and other metals are supported on a β-zeolite may be prepared by carrying a metal such as Cu and Zr onto a β-zeolite by a known method such as an impregnation method and an ion exchange method. A β-zeolite may be prepared by a known method, and may be commercially available.

### 2. Process for Producing a Catalyst for Producing a Liquefied Petroleum Gas According to the Present Invention

A catalyst for producing a liquefied petroleum gas according to the present invention is preferably produced by separately preparing a Cu—Zn-based methanol synthesis catalyst as a methanol synthesis catalyst component and a Cu-supported β-zeolite as a zeolite catalyst component, and then mixing them. By separately preparing a methanol synthesis catalyst component and a zeolite catalyst component, a composition, a structure and a property of each component may be easily optimized for each function.

A Cu—Zn-based methanol synthesis catalyst may be prepared by a known method, as described above. A commercially available Cu—Zn-based methanol synthesis catalyst may be also used.

Some of methanol synthesis catalyst components must be activated by reduction treatment before use. In the present invention, it is not necessarily required to activate a methanol synthesis catalyst component by reduction treatment in advance. The methanol synthesis catalyst component may be activated by subjecting the catalyst for producing a liquefied petroleum gas of the present invention to the reduction treatment, before the beginning of the reaction, after producing the catalyst by mixing a methanol synthesis catalyst component and a zeolite catalyst component, and then molding the mixture. The reduction treatment condition may be determined, depending on some factors such as the type of the methanol synthesis catalyst component, as appropriate.

A Cu-supported β-zeolite as a zeolite catalyst component may be prepared by a known method, as described above.

A catalyst for producing a liquefied petroleum gas according to the present invention may be produced by homogeneously mixing a methanol synthesis catalyst component and a zeolite catalyst component, and then, if necessary, molding the mixture. A procedure of mixing and molding these catalyst components is preferably, but not limited to, a dry method. When mixing and molding these catalyst components by a wet method, there may occur a compound transfer between these catalyst components, for example, neutralization due to transfer of a basic component in a methanol synthesis catalyst component to an acidic site in a zeolite catalyst component, leading to the change in properties, which have been optimized for each function of these catalyst components, and the like. A catalyst may be molded by an extrusion method and a tablet-compression method, for example.

According to the present invention, a methanol synthesis catalyst component and a zeolite catalyst component to be mixed may preferably have a relatively large particle size; specifically a particle size of 100 μm or more. A catalyst for producing a liquefied petroleum gas according to the present invention, which is prepared by mixing a methanol synthesis catalyst component with a particle size of 100 μm or more and a zeolite catalyst component with a particle size of 100 μm or more, and then, if necessary, molding the mixture, may have higher catalytic activity and higher yield of LPG, as compared with a catalyst which is prepared by mixing a methanol synthesis catalyst component with a smaller particle size and a zeolite catalyst component with a smaller particle size.

The particle sizes of a methanol synthesis catalyst component and a zeolite catalyst component to be mixed are more preferably 200 μm or more, particularly preferably 500 μm or more. On the other hand, the particle sizes of a methanol synthesis catalyst component and a zeolite catalyst component to be mixed are preferably 5 mm or less, more preferably 3 mm or less so as to maintain the excellent performance of the mixed catalyst of the present invention.

It is preferred that a methanol synthesis catalyst component and a zeolite catalyst component to be mixed have the same particle size.

In general, each catalyst component is, if necessary, mechanically pulverized to the same particle size of about 0.5 μm to about 2 μm, for example, and then the catalyst components are homogeneously mixed and molded, if necessary, to prepare a mixed catalyst. Alternatively, all of catalyst components are placed into a vessel, and are mixed, while mechanically pulverizing, to prepare a homogeneous mixture with a particle size of about 0.5 μm to about 2 μm, for example, and then the mixture is molded, if necessary.

In contrast, when preparing a catalyst for producing a liquefied petroleum gas according to the present invention by mixing a methanol synthesis catalyst component with a particle size of 100 μm or more and a zeolite catalyst component with a particle size of 100 μm or more, the catalyst may be generally produced as follows. Each catalyst component is molded by a known molding method such as a tablet-compression and an extrusion, and then, if necessary, mechanically pulverized to the same particle size of preferably about 100 μm to about 5 mm. And then, these components are homogeneously mixed. If necessary, the mixture is molded again to prepare a catalyst for producing a liquefied petroleum gas of the present invention.

### 3. Process for Producing a Liquefied Petroleum Gas

Next, there will be described a process for producing a liquefied petroleum gas comprising propane or butane, preferably propane, as a main component in which carbon monoxide and hydrogen are reacted using a catalyst for producing a liquefied petroleum gas according to the present invention as described above.

A reaction temperature is preferably 260° C. or higher, more preferably 270° C. or higher, particularly preferably 275° C. or higher. By controlling a reaction temperature within the above range, higher catalytic activity and higher selectivity for LPG may be achieved, and therefore propane and/or butane may be produced with higher yield.

On the other hand, a reaction temperature is preferably 325° C. or lower, more preferably 315° C. or lower, particularly preferably 310° C. or lower, in the light of the stability and durability of the catalyst.

A reaction pressure is preferably 1.6 MPa or higher, more preferably 1.8 MPa or higher, particularly preferably 1.9 MPa or higher, in the light of the higher catalytic activity.

On the other hand, a reaction pressure is preferably 4.5 MPa or lower, more preferably 4 MPa or lower, in the light of higher selectivity for LPG.

As for a contact time between a starting gas, which contains carbon monoxide and hydrogen, and the catalyst, W/F [ratio of the weight of the catalyst (W; g) to the total flow rate of the starting gas (F; mol/h)] is preferably 2 g·h/mol or more, more preferably 4 g·h/mol or more, in the light of higher conversion of carbon monoxide and higher selectivity for LPG. On the other hand, W/F is preferably 20 g·h/mol or less, more preferably 16 g·h/mol or less, in the light of economical efficiency.

A concentration of carbon monoxide in a gas fed into a reactor is preferably 20 mol % or more, more preferably 25 mol % or more, in the light of ensuring a pressure (partial pressure) of carbon monoxide required for the reaction, and improving a specific productivity of the materials. In addition, a concentration of carbon monoxide in a gas fed into a reactor is preferably 45 mol % or less, more preferably 40 mol % or less, in the light of a further sufficiently high conversion of carbon monoxide.

A concentration of hydrogen in a gas fed into a reactor is preferably 1.2 moles or more, more preferably 1.5 moles or more per one mole of carbon monoxide, in order that carbon monoxide may react more sufficiently. In addition, a concentration of hydrogen in a gas fed into a reactor is preferably 3 moles or less, more preferably 2.5 moles or less per one mole of carbon monoxide, in the light of economical efficiency.

A gas fed into a reactor may contain carbon dioxide in addition to carbon monoxide and hydrogen, which are starting materials of the reaction. By recycling carbon dioxide discharged from the reactor, or by adding the corresponding amount of carbon dioxide, formation of carbon dioxide from carbon monoxide by a shift reaction in the reactor may be substantially reduced or be eliminated.

A gas fed into a reactor may contain water vapor. In addition, a gas fed into a reactor may contain an inert gas such as Ar, and the like.

A gas fed into a reactor may be dividedly fed to the reactor, thereby controlling a reaction temperature.

The reaction may be conducted in a fixed bed, a fluidized bed, a moving bed, a slurry bed, or the like. The reaction may be conducted in a vapor phase, a liquid phase, or a supercritical phase. The reaction mode and the reactor to be used may be preferably selected, taking both of control of a reaction temperature and a regeneration method of the catalyst into account. Examples of the reactor with a fixed bed may include a quench type reactor such as an internal multistage quench type, a multitubular type reactor, a multistage type reactor having a plurality of internal heat exchangers or the like, a multistage cooling radial flow type reactor, a double pipe heat exchange type reactor, an internal cooling coil type reactor, a mixed flow type reactor, and other types of reactors.

The catalyst for producing a liquefied petroleum gas according to the present invention may be diluted with silica, alumina or an inert stable heat conductor and used in order to control a reaction temperature. In addition, the catalyst for producing a liquefied petroleum gas according to the present invention may be applied to the surface of a heat exchanger and used in order to control a reaction temperature.

### 4. Process for Producing a Liquefied Petroleum Gas from a Carbon-Containing Starting Material

According to the present invention, a synthesis gas may be used as a starting gas for producing a liquefied petroleum gas (LPG).

Next, with reference to the drawing, there will be described an embodiment of a process for producing LPG according to the present invention, which comprises producing a synthesis gas from a carbon-containing starting material (synthesis gas production process) and then producing LPG from the obtained synthesis gas using a catalyst of the present invention (liquefied petroleum gas production process).

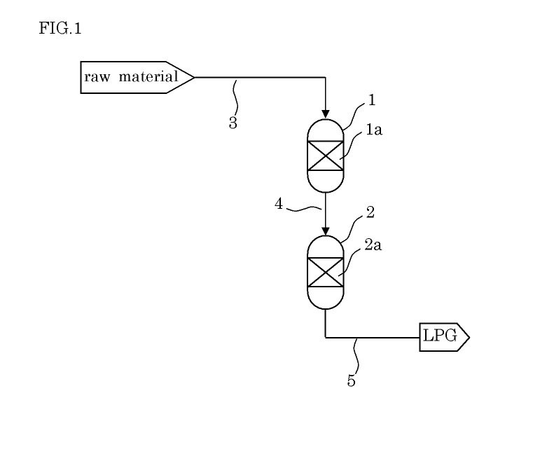

FIG. 1 shows an embodiment of an LPG production apparatus suitable for carrying out a production process for LPG according to the present invention.

First, a natural gas (methane) as a carbon-containing starting material is fed into a reformer 1 via a line 3. In addition, for steam reforming, steam (not shown) is also fed into the line 3. In the reformer 1, there is a reforming catalyst layer 1a comprising a reforming catalyst (a catalyst for producing a synthesis gas). The reformer 1 also has a heating means for supplying heat required for reforming (not shown). In the reformer 1, methane is reformed in the presence of the reforming catalyst to produce a synthesis gas containing hydrogen and carbon monoxide.

The synthesis gas thus produced is fed into a reactor 2 via a line 4. In the reactor 2, there is a catalyst layer 2a comprising a catalyst of the present invention. In the reactor 2, a hydrocarbon gas containing propane or butane as a main component (a lower-paraffin-containing gas) is produced from the synthesis gas in the presence of the catalyst of the present invention.

The hydrocarbon gas thus produced is pressurized and cooled, after optional removal of water and the like, and LPG, which is a product, is obtained from a line 5. Optionally, hydrogen and the like may be removed from the LPG by gas-liquid separation, for example.

A low-boiling component and the like may be separated, by a known method, from the hydrocarbon gas produced in the reactor 2 and recycled into the reformer 1 as a starting material for the synthesis gas production process (reforming process).

A gas obtained by adding carbon dioxide and the like to the synthesis gas produced in the reformer 1 may be fed into the reactor 2. Alternatively, a gas obtained by adding additional hydrogen or carbon monoxide to the synthesis gas produced in the reformer 1 may be fed into the reactor 2. A gas obtained by adjusting its composition by a shift reaction may be fed into the reactor 2.

The LPG production apparatus may be, as necessary, provided with a booster, a heat exchanger, a valve, an instrumentation controller and so on, which are not shown.

<Synthesis Gas Production Process>

In a synthesis gas production process, a synthesis gas is produced from a carbon-containing starting material and at least one selected from the group consisting of H₂O, O₂ and CO₂.

A carbon-containing substance, which can react with at least one selected from the group consisting of H₂O, O₂ and CO₂ to form H₂ and CO, may be used as a carbon-containing starting material. A substance known as a raw material for a synthesis gas may be used as a carbon-containing starting material; for example, lower hydrocarbons such as methane and ethane, a natural gas, a naphtha, a coal, and the like may be used.

According to the present invention, a catalyst is generally used in a synthesis gas production process and a liquefied petroleum gas production process. Therefore, a carbon-containing starting material (a natural gas, a naphtha, a coal and so on) preferably contains less catalyst poisoning components such as sulfur and a sulfur compound. When a carbon-containing starting material contains a catalyst poisoning component, a step of removing the catalyst poisoning component such as devulcanization may be conducted before a synthesis gas production process, if necessary.

A synthesis gas may be produced by reacting the carbon-containing starting material as described above with at least one selected from the group consisting of H₂O, O₂ and CO₂ in the presence of a catalyst for producing a synthesis gas (reforming catalyst).

A synthesis gas may be produced by a known method. When a natural gas (methane) is used as a starting material, for example, a synthesis gas may be produced by a water vapor reforming method, an autothermal reforming method, and the like. In these methods, water vapor required for a water-vapor reforming, oxygen required for an autothermal reforming, and the like may be fed, if necessary. When a coal is used as a starting material, a synthesis gas may be produced using an aerating gasification furnace, and the like.

A shift reactor, for example, may be provided downstream of a reformer, which is a reactor for producing a synthesis gas from the starting materials as described above, so that a synthesis gas composition may be adjusted by a shift reaction (CO+H₂O→CO₂+H₂).

In the present invention, a preferable composition of a synthesis gas produced in a synthesis gas production process is a molar ratio of H₂/CO is 7/3≅2.3 in terms of the stoichiometry for a lower paraffin production, and a ratio of hydrogen to carbon monoxide (H₂/CO; by mole) in a synthesis gas produced is preferably 1.2 to 3. A ratio of hydrogen to carbon monoxide (H₂/CO; by mole) in a synthesis gas is preferably 1.2 or more, more preferably 1.5 or more, in that carbon monoxide may react suitably, because hydrogen is generated by a shift reaction caused by water, which is generated in a conversion reaction from a synthesis gas to LPG. It is preferred that hydrogen is fed only in an amount such that carbon monoxide may react suitably to form a liquefied petroleum gas comprising propane or butane as a main component, and excessive hydrogen may increase the total pressure of a starting gas unnecessarily, leading to a lower economical efficiency. Thus, a ratio of hydrogen to carbon monoxide (H₂/CO; by mole) in a synthesis gas is preferably 3 or less, more preferably 2.5 or less.

A concentration of carbon monoxide in a synthesis gas produced is preferably 20 mol % or more, more preferably 25 mol % or more, in the light of ensuring a pressure (partial pressure) of carbon monoxide suitable for a conversion reaction from a synthesis gas to LPG, and improving a specific productivity of the materials. In addition, a concentration of carbon monoxide in a synthesis gas produced is preferably 45 mol % or less, more preferably 40 mol % or less, in the light of a further sufficiently high conversion of carbon monoxide in a conversion reaction from a synthesis gas to LPG.

A synthesis gas having the composition as described above may be produced by appropriately selecting reaction conditions such as a feeding ratio of a carbon-containing starting material and at least one material selected from the group consisting of steam (water), oxygen and carbon dioxide, a kind of a catalyst for producing a synthesis gas to be used, and the like.

For example, a synthesis gas may be produced using a gas whose composition is steam/methane (molar ratio) of 1 and carbon dioxide/methane (molar ratio) of 0.4 as a starting gas under the operation conditions of a reaction temperature (an outlet temperature of a catalyst layer) of 800 to 900° C., a reaction pressure of 1 to 4 MPa, a gas space velocity (GHSV) of 2000 hr⁻¹, in an external heating multitubular tubular-reactor type apparatus filled with a catalyst, a Ru or Rh/a sintered magnesia having the smaller surface area.

When using steam for reforming in a synthesis gas production, a ratio of steam/raw material carbon (S/C) is preferably 1.5 or less, more preferably 0.8 to 1.2, in the light of an energy efficiency. Meanwhile, such a low SIC value may lead to the considerable possibility of carbon precipitation formation.

When producing a synthesis gas with a low S/C, it may be preferable to use a catalyst which have a good activity of forming a synthesis gas and a low activity of forming a carbon precipitation, as described in, for example, WO 98/46524, JP-A-2000-288394 and JP-A-2000-469. Hereinafter, such a catalyst will be described.

The catalyst described in WO 98/46524 is a catalyst in which at least one catalyst metal selected from rhodium, ruthenium, iridium, palladium and platinum is supported on a support composed of a metal oxide, which has a specific surface area of 25 m²/g or less, an electronegativity of a metal ion in the support metal oxide of 13.0 or less, and the amount of the supported catalyst metal of 0.0005 to 0.1 mol % relative to the support metal oxide in terms of metal. In the light of prevention of carbon precipitation, the electronegativity is preferably 4 to 12 and the specific surface area of the catalyst is preferably 0.01 to 10 m²/g.

An electronegativity of a metal ion in the metal oxide is defined by the following equation:

Xi=(1+2i)Xo
wherein Xi represents an electronegativity of the metal ion; Xo represents an electronegativity of the metal; and i represents an electronic number of the metal ion.

When the metal oxide is a composite metal oxide, an average electronegativity of the metal ions is used, and the value is the sum total of electronegativity of the each metal ion in the composite metal oxide multiplied by a mole fraction of each oxide.

An electronegativity of a metal (Xo) is a Pauling's electronegativity. Pauling's electronegativities are listed in Table 15.4 in “Moore Physical Chemistry (latter volume) (4th edition), translated by Ryoichi Fujishiro, Tokyo Kagaku Dozin Co., Ltd., p. 707 (1974)”. An electronegativity of a metal ion (Xi) in a metal oxide is detailed in, for example, “Shokubai Koza (Lectures on Catalyst), Vol. 2, p. 145, ed. the Catalysis Society of Japan (1985)”.

Examples of the metal oxide in the catalyst include those containing at least one metal such as Mg, Ca, Ba, Zn, Al, Zr and La. An example of such a metal oxide is magnesia (MgO).

In a process in which methane and steam are reacted (steam reforming), the reaction is represented by the following formula (I):

CH₄+H₂O⇄3H₂+CO (i)

In a process in which methane and carbon dioxide are reacted (CO₂ reforming), the reaction is represented by the following formula (ii):

CH₄+CO₂⇄2H₂+2CO (ii)

In a process in which methane, steam and carbon dioxide are reacted (steam/CO₂ mixed reforming), the reaction is represented by the following formula

3CH₄+2H₂O+CO₂⇄8H₂+4CO(iii)

In steam reforming using the catalyst as described above, a reaction temperature is preferably 600 to 1200° C., more preferably 600 to 1000° C., and a reaction pressure is preferably 0.098 MPaG to 3.9 MPaG, more preferably 0.49 MPaG to 2.9 MPaG (G indicates that the value is a gauge pressure). When the steam reforming is conducted with a fixed bed, a gas space velocity (GHSV) is preferably 1,000 to 10,000 hr⁻¹, more preferably 2,000 to 8,000 hr⁻¹. A rate of steam to a carbon-containing starting material is preferably 0.5 to 2 moles, more preferably 0.5 to 1.5 moles, further preferably 0.8 to 1.2 moles of steam (H₂O) per one mole of carbon in the carbon-containing starting material (excluding CO₂).

In CO₂ reforming using the catalyst as described above, a reaction temperature is preferably 500 to 1200° C., more preferably 600 to 1000° C., and a reaction pressure is preferably 0.49 MPaG to 3.9 MPaG, more preferably 0.49 MPaG to 2.9 MPaG. When the CO₂ reforming is conducted with a fixed bed, a gas space velocity (GHSV) is preferably 1,000 to 10,000 hr⁻¹, more preferably 2,000 to 8,000 hr⁻¹. A rate of CO₂ to a carbon-containing starting material is preferably 20 to 0.5 moles, more preferably 10 to 1 moles of CO₂ per one mole of carbon in the carbon-containing starting material (excluding CO₂).

When a carbon-containing starting material is reacted with a mixture of steam and CO₂ using the catalyst as described above to produce a synthesis gas (i.e. steam/CO₂ mixed reforming is conducted), there are no restrictions to a ratio of steam to CO₂, but a ratio of H₂O/CO₂ (molar ratio) is generally 0.1 to 10. A reaction temperature is preferably 550 to 1200° C., more preferably 600 to 1000° C., and a reaction pressure is preferably 0.29 MPaG to 3.9 MPaG, more preferably 0.49 MPaG to 2.9 MPaG. When the reaction is conducted with a fixed bed, a gas space velocity (GHSV) is preferably 1,000 to 10,000 hr⁻¹, more preferably 2,000 to 8,000 hr⁻¹. A rate of steam to a carbon-containing starting material is preferably 0.5 to 2 moles, more preferably 0.5 to 1.5 moles, further preferably 0.5 to 1.2 moles of steam (H₂O) per one mole of carbon in the carbon-containing starting material (excluding CO₂).

The catalyst described in JP-A-2000-288394 is composed of a composite oxide having a composition represented by the following formula (I), characterized in that M¹ and Co are highly dispersed in the composite oxide:

a¹M¹-b¹Co-c¹Mg-d¹Ca-e¹O (I)

wherein a¹, b¹, c¹, d¹ and e¹ are mole fractions, provided that a¹+b¹+c¹+d¹=1, 0.0001≦a¹≦0.10, 0.0001≦b¹≦0.20, 0.70≦(c¹+d¹)≦0.9998, 0<c¹≦0.9998, 0≦d¹<0.9998, and e¹ is a number required for maintaining the charge balance of the elements and oxygen;

M¹ is at least one element selected from Group 6A elements, Group 7A elements, Group 8 transition elements except Co, Group 1B elements, Group 2B elements, Group 4B elements and lanthanoid elements in the Periodic Table.

The catalyst described in JP-A-2000-469 is composed of a composite oxide having a composition represented by the following formula (II), characterized in that M² and Ni are highly dispersed in the composite oxide:

a²M²-b²Ni-c²Mg-d²Ca-e²O (II)

wherein a², b², c², d² and e² are mole fractions, provided that a²+b²+c²+d²=1, 0.0001≦a²≦0.10, 0.0001≦b²≦0.10, 0.80≦(c²+d²)≦0.9998, 0<c²≦0.9998, 0≦d²<0.9998, and e² is a number required for maintaining the charge balance of the elements and oxygen;

M² is at least one element selected from Group 3B elements, Group 4A elements, Group 6B elements, Group 7B elements, Group 1A elements and lanthanoid elements in the Periodic Table.

These catalysts may be used in the same way as the catalyst described in WO 98/46524.

A reforming reaction of a carbon-containing starting material, i.e. a reaction for producing a synthesis gas, is not limited to the methods as described above, and may be conducted in accordance with any of other known methods. A reforming reaction of a carbon-containing starting material may be conducted in various types of reactors, but is preferably conducted in a fixed bed or a fluidized bed.

<Liquefied Petroleum Gas Production Process>

In a liquefied petroleum gas production process, a lower-paraffin-containing gas, which comprises propane or butane as a main component of the hydrocarbon contained therein, is produced from the synthesis gas obtained in the synthesis gas production process as described above, using a catalyst for producing a liquefied petroleum gas according to the present invention. And then, water is separated from the lower-paraffin-containing gas produced, as necessary, and subsequently a low-boiling component having a lower boiling point or a lower sublimation point than the boiling point of propane (unreacted starting materials, hydrogen and carbon monoxide; by-products, carbon dioxide, ethane, ethylene and methane; and so on) and a high-boiling component having a higher boiling point than the boiling point of butane (by-products, high-boiling paraffin gases; and so on) are separated from the lower-paraffin-containing gas, as necessary, to obtain a liquefied petroleum gas (LPG) comprising propane or butane as a main component. If necessary, the gas may be pressurized and/or cooled so as to obtain a liquefied petroleum gas.

In a liquefied petroleum gas production process, carbon monoxide and hydrogen are reacted in the presence of the catalyst for producing a liquefied petroleum gas of the present invention as described above, to produce a paraffin comprising propane or butane as a main component, preferably a paraffin comprising propane as a main component.

In this process, a gas fed into a reactor is the synthesis gas produced in the synthesis gas production process as described above. The gas fed into a reactor may be a gas obtained by adding carbon monoxide, hydrogen and/or other components (carbon dioxide, water vapor, etc.), if necessary, to the synthesis gas produced in the synthesis gas production process as described above. Alternatively, the gas fed into a reactor may be a gas obtained by separating a certain component, as necessary, from the synthesis gas produced in the synthesis gas production process as described above.

The reaction (LPG synthesis reaction) to form a lower-paraffin-containing gas, using a catalyst according to the present invention, may be conducted under the reaction conditions as described above.

A lower-paraffin-containing gas produced in the liquefied petroleum gas production process comprises a hydrocarbon containing propane or butane as a main component. In the light of liquefaction properties, it is preferable that the total content of propane and butane is higher in a lower-paraffin-containing gas. According to the present invention, there may be obtained a lower-paraffin-containing gas having a content of propane and butane of 60% or more, preferably 70% or more, more preferably 75% or more (including 100%) on the basis of carbon to the hydrocarbon contained therein, in total.

Furthermore, a lower-paraffin-containing gas produced in the liquefied petroleum gas production process preferably contains more propane, as compared with butane, in the light of inflammability and vapor pressure properties.

A lower-paraffin-containing gas produced in a liquefied petroleum gas production process generally comprises water; a low-boiling component having a lower boiling point or a lower sublimation point than the boiling point of propane; and a high-boiling component having a higher boiling point than the boiling point of butane. Examples of a low-boiling component include ethane, methane and ethylene, which are by-products; carbon dioxide which is formed by a shift reaction; and hydrogen and carbon monoxide, which are unreacted starting materials. Examples of a high-boiling component include high-boiling paraffins (e.g., pentane, hexane and so on), which are by-products.

Accordingly, water, a low-boiling component and a high-boiling component may be separated from a lower-paraffin-containing gas produced, as necessary, to obtain a liquefied petroleum gas (LPG) comprising propane or butane as a main component.

Separations of water, a low-boiling component and a high-boiling component may be conducted in accordance with a known method.

Water may be separated by, for example, liquid-liquid separation.

A low-boiling component may be separated by, for example, gas-liquid separation, absorption separation or distillation; more specifically, gas-liquid separation at an ambient temperature under increased pressure, absorption separation at an ambient temperature under increased pressure, gas-liquid separation with cooling, absorption separation with cooling, or combination thereof. Alternatively, for separation of a low-boiling component, membrane separation or adsorption separation may be conducted, and one of these separations in combination with gas-liquid separation, absorption separation or distillation may be conducted. A gas recovery process commonly employed in an oil factory (described in “Oil Refining Processes”, ed. The Japan Petroleum Institute, Kodansha Scientific, 1998, pp. 28-32) may be employed for separation of a low-boiling component.

A preferable method of separation of a low-boiling component may be an absorption process in which a liquefied petroleum gas comprising propane or butane as a main component is absorbed into an absorbent liquid such as a high-boiling paraffin gas having a higher boiling point than butane, and a gasoline.

A high-boiling component may be separated by, for example, gas-liquid separation, absorption separation or distillation.

For consumer use, a content of a low-boiling component in the LPG is preferably reduced to 5 mol % or less (including 0 mol %) by separation, for example, in the light of safety in use.

The total content of propane and butane in the LPG thus produced may be 90 mol % or more, more preferably 95 mol % or more (including 100 mol %).

According to the present invention, a low-boiling component separated from the lower-paraffin-containing gas may be recycled as a starting material for the synthesis gas production process.

A low-boiling component separated from the lower-paraffin-containing gas comprises substances which may be used as starting materials for a synthesis gas production process; for example, methane, ethane, ethylene and so on. In addition, carbon dioxide in the low-boiling component may be converted to a synthesis gas by a CO₂ reforming reaction. Furthermore, a low-boiling component may comprise hydrogen and carbon monoxide, which are unreacted starting materials. Accordingly, the low-boiling component separated from the lower-paraffin-containing gas may be recycled as a starting material for a synthesis gas production process, leading to an increase in productivity per starting material.

The whole low-boiling components separated from a lower-paraffin-containing gas may be recycled to a synthesis gas production process. Alternatively, part of the low-boiling components may be removed outside the system, while the rest may be recycled to a synthesis gas production process. A desired component may be separated from the low-boiling components and recycled to a synthesis gas production process.

In a synthesis gas production process, a content of a low-boiling component in a gas fed into a reformer (i.e. reactor), in other words, a content of a recycled material may be determined as appropriate, and it may be, for example, 40 to 75 mol %.

Any known technique, e.g. providing a recycle line with a pressurization means may be employed as appropriate, to recycle a low-boiling component.

### EXAMPLES

The following will describe the present invention in more detail with reference to Examples. However, the present invention is not limited to these Examples.

### Example 1

### Preparation of Catalyst

A Cu—Zn—Al—Cr composite oxide (average particle size: about 0.35 mm to about 0.7 mm) was used as a Cu—Zn-based methanol synthesis catalyst, which is a methanol synthesis catalyst component.

As a Cu-supported β-zeolite, which is a zeolite catalyst component, 0.5 wt % of Cu was supported on a commercially available proton-type β-zeolite with a SiO₂/Al₂O₃ ratio of 37 (produced by ZEOLYST INTERNATIONAL Inc.) by an ion exchange method as follows, to prepare a Cu-supported β-zeolite (average particle size: about 0.35 mm to about 0.7 mm).

First, 0.57 g of Cu(NO₃)₂.3H₂O was dissolved in 150 mL of ion-exchanged water to prepare a Cu-containing solution (concentration: 0.067 wt %). And then, 2.0 g of βzeolite was added to the resulting Cu-containing solution, and the mixture was heated and stirred at 65° C. for 8 hours. The resulting material was filtrated and washed with ion-exchanged water three times.

The Cu-supported β-zeolite thus obtained was dried at 120° C. for 10 hours, and then calcined at 500° C. for 4 hours. Subsequently, the Cu-supported β-zeolite was mechanically pulverized, and molded by a tablet-compression and sized to give a Cu-supported β-zeolite having an average particle size of 0.35 to 0.7 mm.

And then, the Cu—Zn-based methanol synthesis catalyst (hereinafter, also referred to as “Cu—Zn”) and the Cu-supported β-zeolite thus prepared (hereinafter, also referred to as “0.5% Cu-β-37”) were homogeneously mixed at a weight ratio of Cu—Zn:0.5% Cu-β-37=1:1, to give a catalyst for producing a liquefied petroleum gas.

(Production of LPG)

In a tubular reactor with an inner diameter of 6 mm was placed 1 g of the catalyst thus prepared. The catalyst was subjected to reduction treatment under a hydrogen stream at 300° C. for 3 hours prior to the reaction.

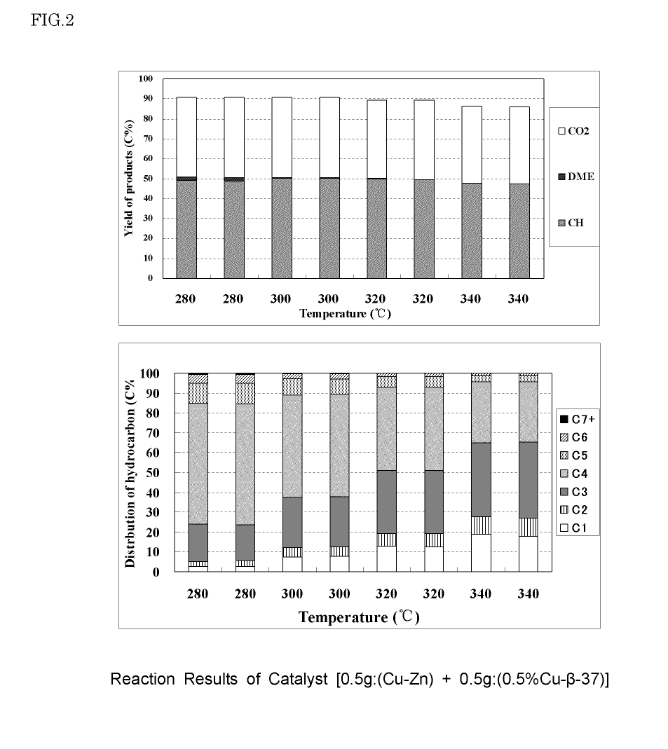

After reduction treatment of the catalyst, a starting gas having the composition of H₂:CO:Ar=64.56:32.4:3.0 (molar ratio) was passed through the catalyst layer at various reaction temperatures (from 280° C. to 340° C.) shown in FIG. 2, a reaction pressure of 2.0 MPa, and a W/F of 8.9 μl/mol, to carry out the LPG production reaction. A gas chromatography was used for the analysis of the product.

FIG. 2 shows the conversion of carbon monoxide into hydrocarbon (CH), the conversion of carbon monoxide into carbon dioxide by shift reaction, and the composition of the produced hydrocarbon at a reaction time of 3 hours. The catalyst exhibited higher catalytic activity and higher selectivity for LPG, particularly at low temperatures.

### Example 2

### Preparation of Catalyst

A catalyst was prepared in the same way as Example 1, except that a proton-type β-zeolite with a SiO₂/Al₂O₃ ratio of 350 (produced by ZEOLYST INTERNATIONAL Inc.) was used as a support (β-zeolite) for the Cu-supported β-zeolite. Hereinafter, the Cu-supported β-zeolite thus prepared was also referred to as “0.5% Cu-β-350”.

(Production of LPG)

Using the prepared catalyst, the LPG production reaction and gas chromatographic analysis of the product were carried out in the same way as Example 1.

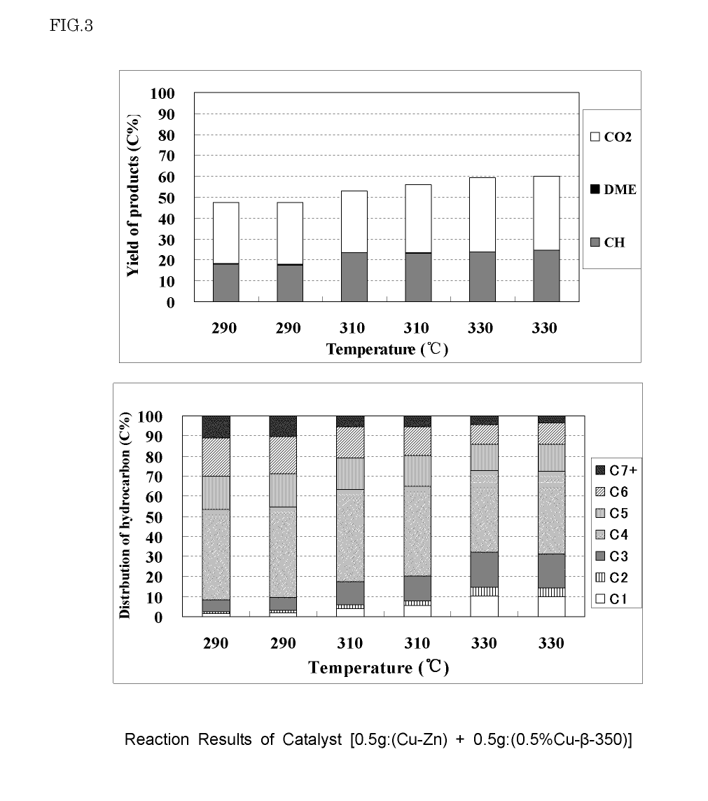

FIG. 3 shows the conversion of carbon monoxide into hydrocarbon (CH), the conversion of carbon monoxide into carbon dioxide by shift reaction, and the composition of the produced hydrocarbon at a reaction time of 3 hours. The catalyst comprising the β-zeolite with a SiO₂/Al₂O₃ ratio of 350 exhibited lower catalytic activity and higher production of higher hydrocarbon (C5+) in particular, as compared with the catalyst comprising the fβ-zeolite with a SiO₂/Al₂O₃ ratio of 37.

### Example 3

### Preparation of Catalyst

A catalyst was prepared in the same way as Example 1, except that the amount of Cu in the Cu-supported β-zeolite was 5.0 wt %. Hereinafter, the Cu-supported β-zeolite thus prepared was also referred to as “5.0% Cu/β-37”.

(Production of LPG)

Using the prepared catalyst, the LPG production reaction and gas chromatographic analysis of the product were carried out in the same way as Example 1 at various reaction temperatures (from 267° C. to 330° C.), a reaction pressure of 2.0 MPa, and a W/F of 8.9 g·h/mol, except that the catalyst was subjected to reduction treatment at 300° C. for 4 hours.

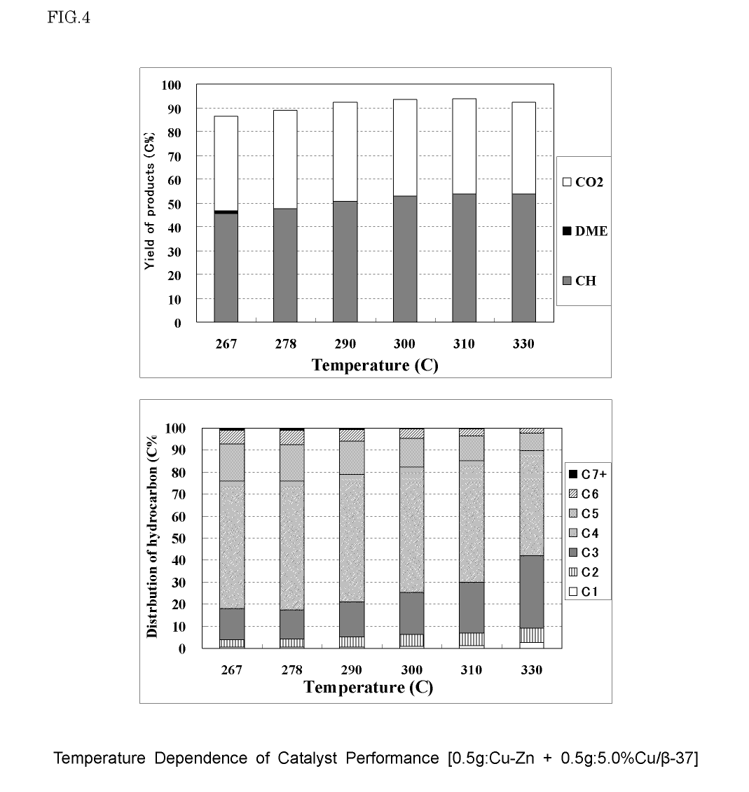

FIG. 4 shows the conversion of carbon monoxide into hydrocarbon (CH), the conversion of carbon monoxide into carbon dioxide by shift reaction, and the composition of the produced hydrocarbon at a reaction time of 3 hours. The catalyst exhibited higher conversion of carbon monoxide and higher selectivity for LPG as the reaction temperature increased, and very low production of methane at high temperatures.

### Example 4

### Preparation of Catalyst

A catalyst was prepared in the same way as Example 1, except that the amount of Cu in the Cu-supported β-zeolite was 10 wt %. Hereinafter, the Cu-supported β-zeolite thus prepared was also referred to as “10% Cu/β-37”.

(Production of LPG)

Using the prepared catalyst, the LPG production reaction and gas chromatographic analysis of the product were carried out in the same way as Example 1 at various reaction temperatures (from 273° C. to 335° C.), a reaction pressure of 2.0 MPa, and a W/F of 8.9 g·h/mol, except that the catalyst was subjected to reduction treatment at 300° C. for 4 hours.

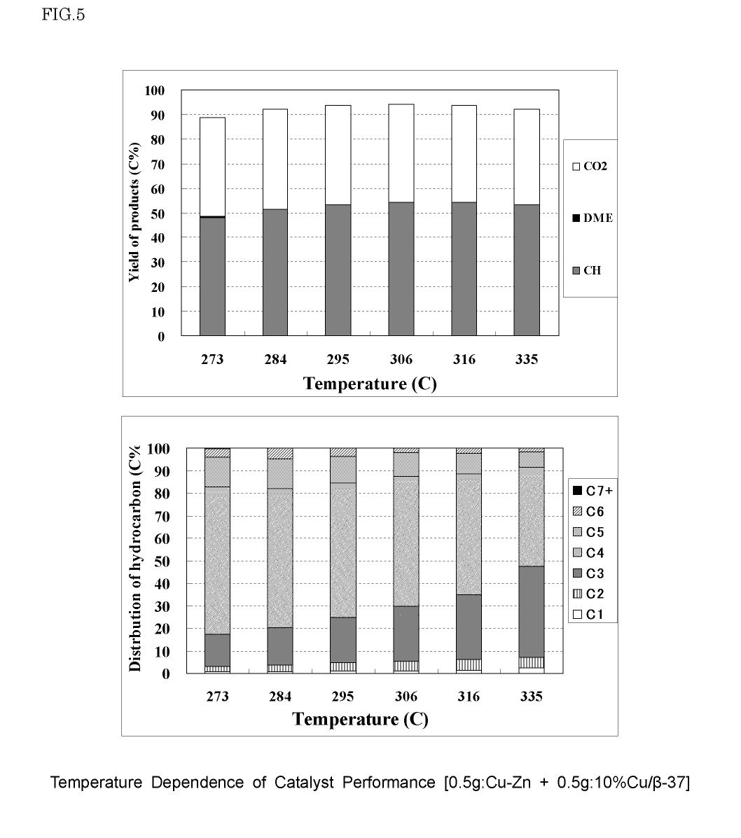

FIG. 5 shows the conversion of carbon monoxide into hydrocarbon (CH), the conversion of carbon monoxide into carbon dioxide by shift reaction, and the composition of the produced hydrocarbon at a reaction time of 3 hours. The catalyst wherein the amount of Cu in the Cu-supported β-zeolite was 10 wt % exhibited further higher selectivity for LPG, although the conversion of carbon monoxide was not enhanced, and very low production of methane.

### Example 5

### Production of LPG

Using the catalyst (Cu—Zn+5.0% Cu/β-37) prepared in the same way as Example 3, the LPG production reaction and gas chromatographic analysis of the product were carried out in the same way as Example 3 at various reaction pressures (from 2.0 MPa to 5.0 MPa), except that the reaction temperature was 300° C.

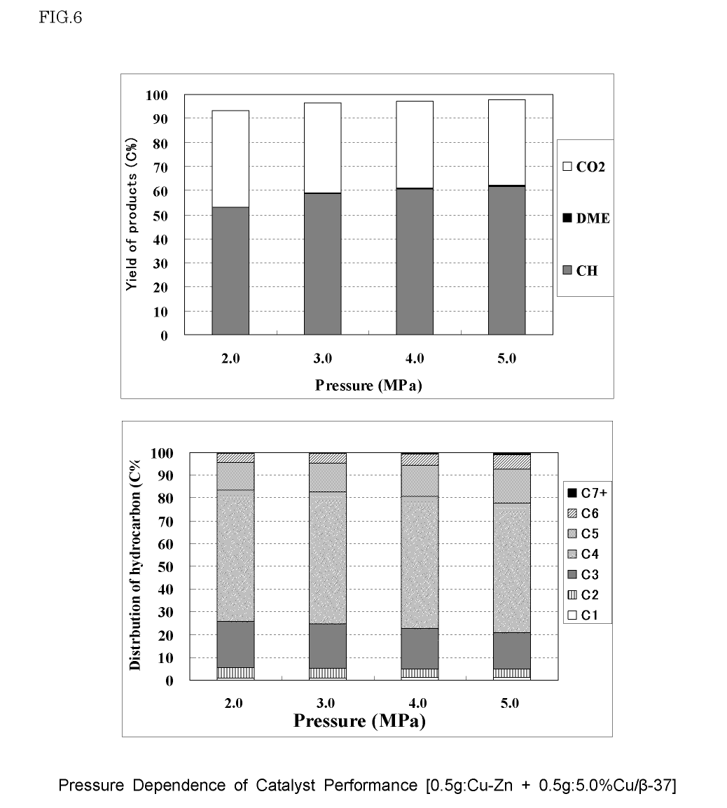

FIG. 6 shows the conversion of carbon monoxide into hydrocarbon (CH), the conversion of carbon monoxide into carbon dioxide by shift reaction, and the composition of the produced hydrocarbon at a reaction time of 3 hours. The catalyst exhibited higher conversion of carbon monoxide and lower selectivity for LPG as the reaction pressure increased.

### Example 6

### Production of LPG

Using the catalyst (Cu—Zn+5.0% Cu/β-37) prepared in the same way as Example 3, the LPG production reaction and gas chromatographic analysis of the product were carried out in the same way as Example 3 at various W/Fs (from 2.6 g·h/mol to 15.0 g·h/mol), except that the reaction temperature was 300° C.

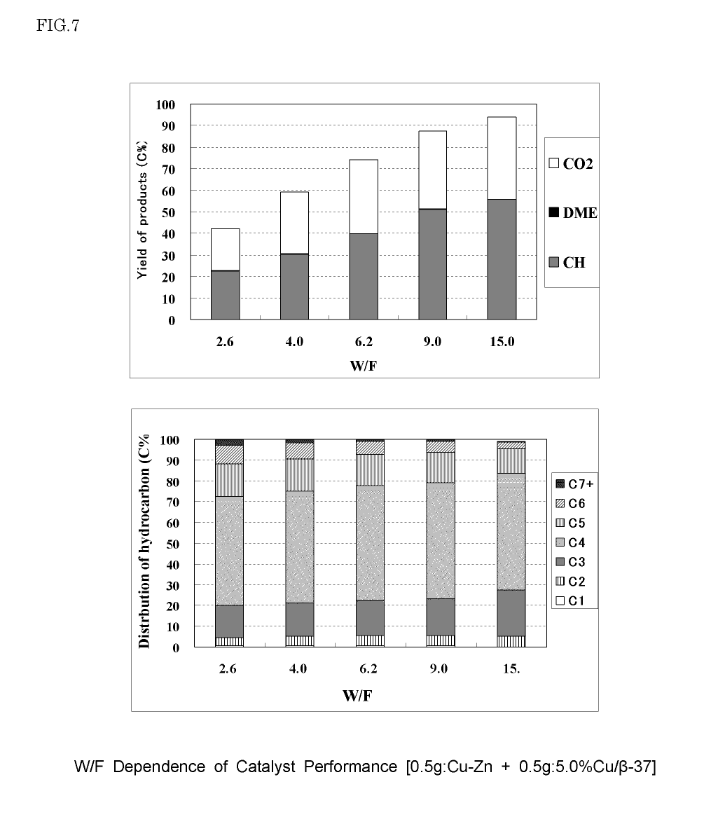

FIG. 7 shows the conversion of carbon monoxide into hydrocarbon (CH), the conversion of carbon monoxide into carbon dioxide by shift reaction, and the composition of the produced hydrocarbon at a reaction time of 3 hours. The catalyst exhibited much higher conversion of carbon monoxide and higher selectivity for LPG as the contact time between the starting gas and the catalyst was longer.

### Example 7

### Production of LPG

Using the catalyst (Cu—Zn+5.0% Cu/β-37) prepared in the same way as Example 3, the LPG production reaction and gas chromatographic analysis of the product were carried out in the same way as Example 3, except that the reaction temperature was 300° C. (Reaction conditions: reaction temperature: 300° C.; reaction pressure: 2.0 MPa; W/F: 8.9 g·h/mol).

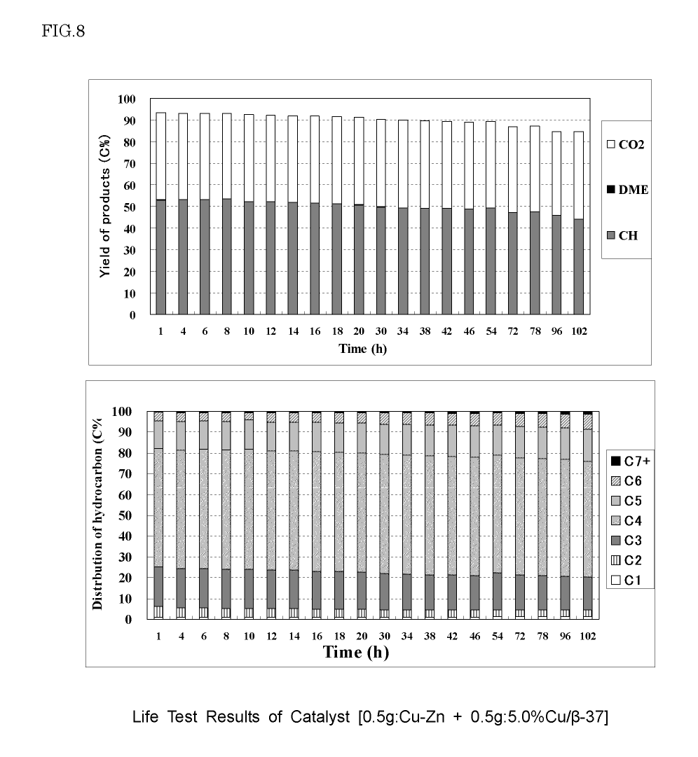

FIG. 8 shows the conversion of carbon monoxide into hydrocarbon (CH), the conversion of carbon monoxide into carbon dioxide by shift reaction, and the composition of the produced hydrocarbon over time. The catalyst exhibited higher catalytic activity and higher selectivity for LPG, and less deterioration over time and higher stability.

### Example 8

### Production of LPG

Using the catalyst (Cu—Zn+10% Cu/β-37) prepared in the same way as Example 4, the LPG production reaction and gas chromatographic analysis of the product were carried out in the same way as Example 7, except that the catalyst was subjected to reduction treatment at 290° C. for 4 hours.

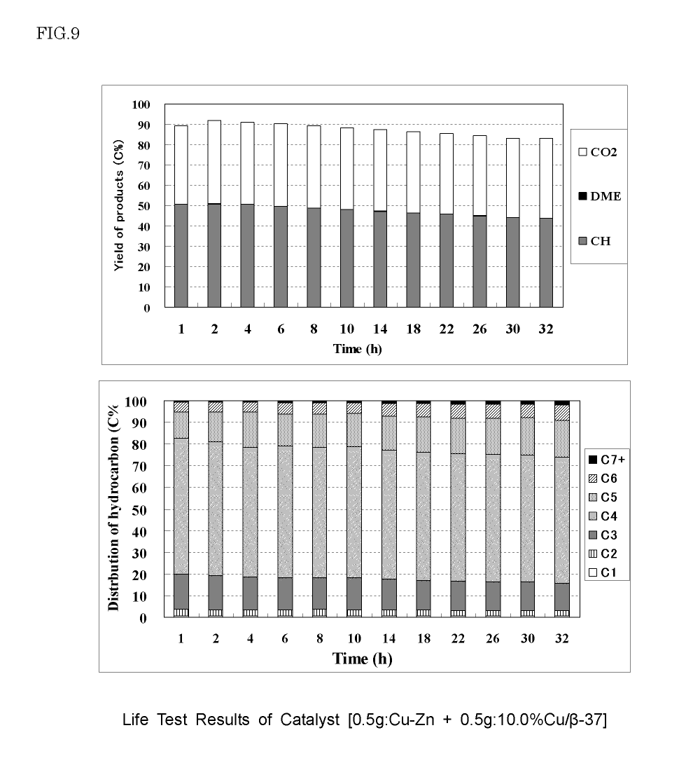

FIG. 9 shows the conversion of carbon monoxide into hydrocarbon (CH), the conversion of carbon monoxide into carbon dioxide by shift reaction, and the composition of the produced hydrocarbon over time. The catalyst wherein the amount of Cu in the Cu-supported β-zeolite was 10 wt % exhibited higher initial activity but the catalytic activity rapidly decreased, as compared with the catalyst wherein the amount of Cu in the Cu-supported β-zeolite was 5 wt %.

### Example 9

### Preparation of Catalyst

A catalyst was prepared in the same way as Example 1, except that the amount of Cu in the Cu-supported β-zeolite was 2.0 wt %. Hereinafter, the Cu-supported β-zeolite thus prepared was also referred to as “2.0% Cu/13-37”.

(Production of LPG)

Using the prepared catalyst (Cu—Zn+2.0% Cu/β-37), the LPG production reaction and gas chromatographic analysis of the product were carried out in the same way as Example 7, except that the catalyst was subjected to reduction treatment at 290° C. for 4 hours.

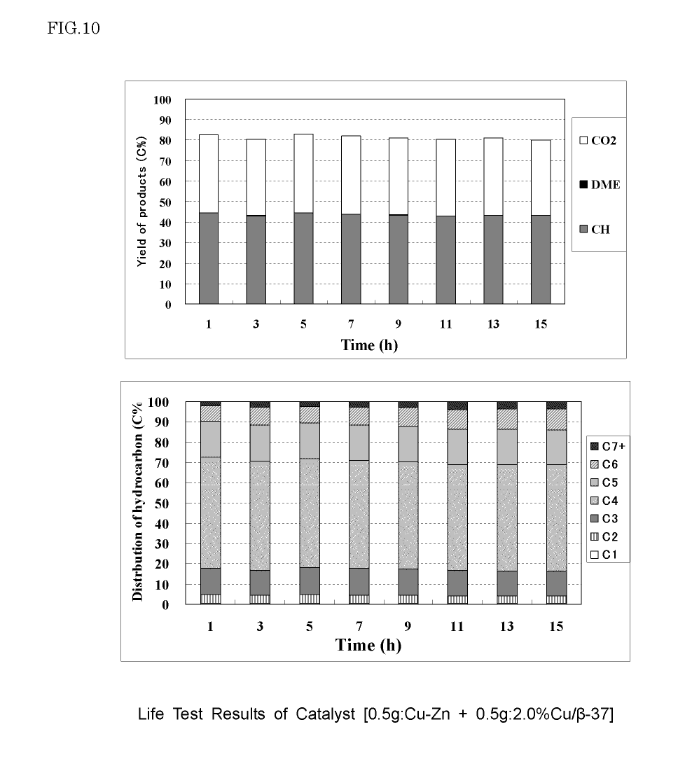

FIG. 10 shows the conversion of carbon monoxide into hydrocarbon (CH), the conversion of carbon monoxide into carbon dioxide by shift reaction, and the composition of the produced hydrocarbon over time. The catalyst wherein the amount of Cu in the Cu-supported β-zeolite was 2 wt % exhibited high stability but lower selectivity for LPG, as compared with the catalyst wherein the amount of Cu in the Cu-supported β-zeolite was 5 wt %.

### Example 10

### Preparation of Catalyst

A catalyst was prepared in the same way as Example 1, except that a Cu-supported β-zeolite [hereinafter, also referred to as “(5.0% Cu+2.5% Zn)/β-37”] in which 5.0 wt % of Cu and 2.5 wt % of Zn were supported on a β-zeolite with a SiO₂/Al₂O₃ ratio of 37 was used as a zeolite catalyst component.

The Cu-supported β-zeolite [(5.0% Cu+2.5% Zn)/β-37] was prepared as follows.

First, 0.57 g of Cu(NO₃)₂·3H₂O and 0.22 g of Zn(NO₃)₂.6H₂O were dissolved in 150 mL of ion-exchanged water to prepare a Cu, Zn-containing solution (Cu concentration: 0.067 wt %; Zn concentration: 0.034 wt %). And then, 2.0 g of proton-type β-zeolite with a SiO₂/Al₂O₃ ratio of 37 (produced by ZEOLYST INTERNATIONAL Inc.) was added to the resulting Cu, Zn-containing solution, and the mixture was heated and stirred at 65° C. for 8 hours. The resulting material was filtrated and washed with ion-exchanged water three times.

The Cu-supported β-zeolite thus obtained was dried at 120° C. for 10 hours, and then calcined at 500° C. for 4 hours. Subsequently, the Cu-supported β-zeolite was mechanically pulverized, and molded by a tablet-compression and sized to give a Cu-supported β-zeolite having an average particle size of 0.35 to 0.7 mm.

(Production of LPG)

Using the prepared catalyst [Cu—Zn+(5.0% Cu+2.5% Zn)/β-37], the LPG production reaction and gas chromatographic analysis of the product were carried out in the same way as Example 7, except that the catalyst was subjected to reduction treatment at 280° C. for 4 hours, and the reaction temperature was 290° C.

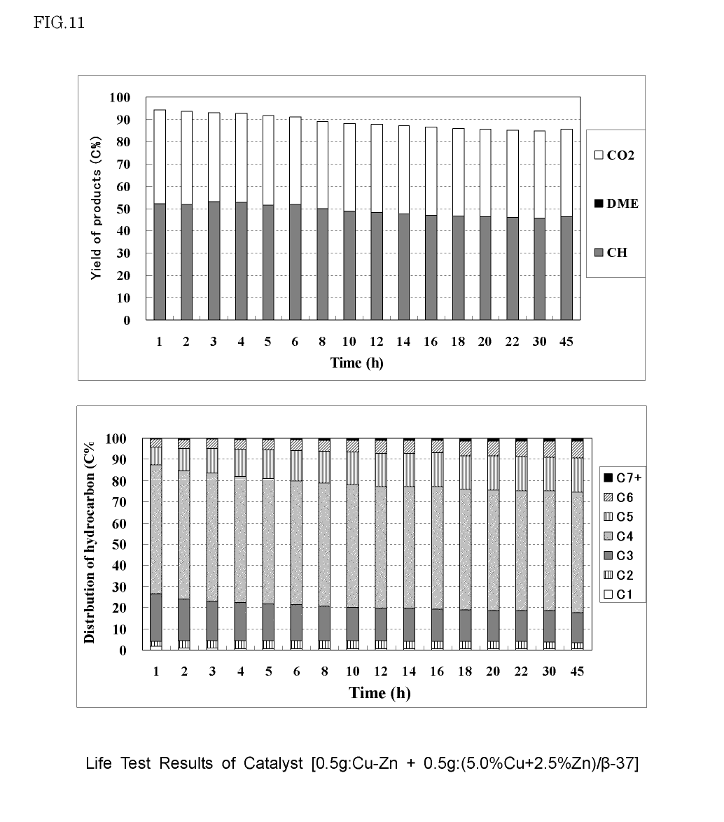

FIG. 11 shows the conversion of carbon monoxide into hydrocarbon (CH), the conversion of carbon monoxide into carbon dioxide by shift reaction, and the composition of the produced hydrocarbon over time. The catalyst wherein Zn in addition to Cu was supported on the 62-zeolite did not exhibit a remarkable effect and the catalytic activity relatively rapidly decreased.

### Example 11

### Preparation of Catalyst

A catalyst was prepared in the same way as Example 1, except that a Cu-supported β-zeolite [hereinafter, also referred to as “(5.0% Cu+2.5% Zr)/β-37”] in which 5.0 wt % of Cu and 2.5 wt % of Zr were supported on a β-zeolite with a SiO₂/Al₂O₃ ratio of 37 was used as a zeolite catalyst component.

The Cu-supported β-zeolite [(5.0% Cu+2.5% Zr)/β-37] was prepared as follows.

First, 0.57 g of Cu(NO₃)₂.3H₂O and 0.147 g of ZrO(NO₃)₂ were dissolved in 150 mL of ion-exchanged water to prepare a Cu, Zr-containing solution (Cu concentration: 0.067 wt %; Zr concentration: 0.034 wt %). And then, 2.0 g of proton-type β-zeolite with a SiO₂/Al₂O₃ ratio of 37 (produced by ZEOLYST INTERNATIONAL Inc.) was added to the resulting Cu, Zr-containing solution, and the mixture was heated and stirred at 65° C. for 8 hours. The resulting material was filtrated and washed with ion-exchanged water three times.

The Cu-supported β-zeolite thus obtained was dried at 120° C. for 10 hours, and then calcined at 500° C. for 4 hours. Subsequently, the Cu-supported β-zeolite was mechanically pulverized, and molded by a tablet-compression and sized to give a Cu-supported β-zeolite having an average particle size of 0.35 to 0.7 mm.

(Production of LPG)

Using the prepared catalyst [Cu—Zn+(5.0% Cu+2.5% Zr)/β-37], the LPG production reaction and gas chromatographic analysis of the product were carried out in the same way as Example 7, except that the catalyst was subjected to reduction treatment at 280° C. for 4 hours, and the reaction temperature was 290° C.

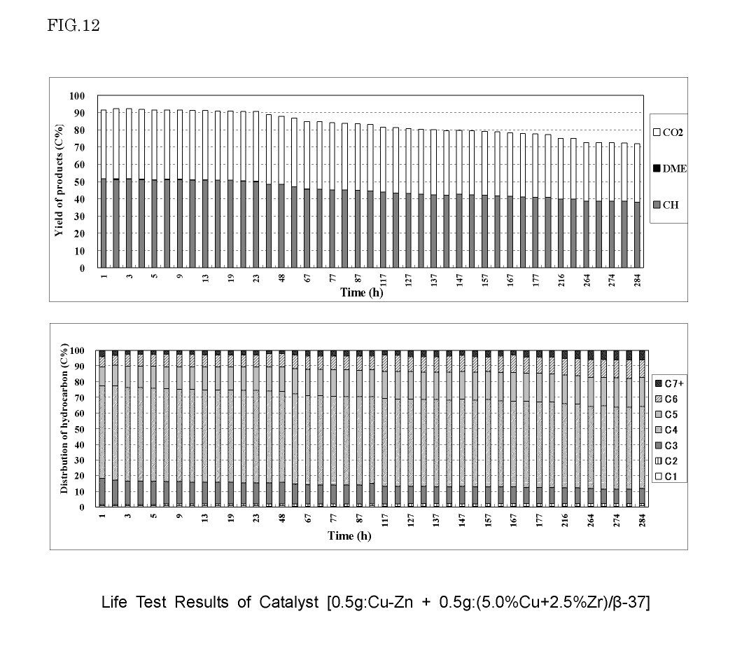

FIG. 12 shows the conversion of carbon monoxide into hydrocarbon (CH), the conversion of carbon monoxide into carbon dioxide by shift reaction, and the composition of the produced hydrocarbon over time. The catalyst wherein Zr in addition to Cu was supported on the β-zeolite exhibited higher stability.

### Example 12

### Preparation of Catalyst

A catalyst was prepared in the same way as Example 11, except that a Cu—Zn-based methanol synthesis catalyst [hereinafter, also referred to as “Cu—Zn+2.5% Cr”] in which 2.5 wt % of Cr was supported on a self-made Cu—Zn-based methanol synthesis catalyst (Cu—Zn—Al composite oxide) by an impregnation method was used as a methanol synthesis catalyst component.

The Cu—Zn-based methanol synthesis catalyst [Cu—Zn+2.5% Cr] as prepared as follows.

First, 0.58 g of Cr(NO₃)₃.9H₂O was dissolved in 3.5 mL of ion-exchanged water to prepare a Cr-containing solution (concentration: 2.1 wt %). And then, 3 g of Cu—Zn-based methanol synthesis catalyst (Cu—Zn—Al composite oxide) was added to the resulting Cr-containing solution, and impregnated with the Cr-containing solution for 3 hours. The resulting Cu—Zn-based methanol synthesis catalyst impregnated with the Cr-containing solution was dried at 120° C. for 10 hours, and then calcined at 500° C. for 4 hours. Subsequently, the Cr-supported Cu—Zn-based methanol synthesis catalyst was mechanically pulverized, and molded by a tablet-compression and sized to give a Cr-supported Cu—Zn-based methanol synthesis catalyst having an average particle size of 0.35 to 0.7 mm.

(Production of LPG)

Using the prepared catalyst [(Cu—Zn+2.5% Cr)+(5.0% Cu+2.5% Zr)/β-37], the LPG production reaction and gas chromatographic analysis of the product were carried out in the same way as Example 11.

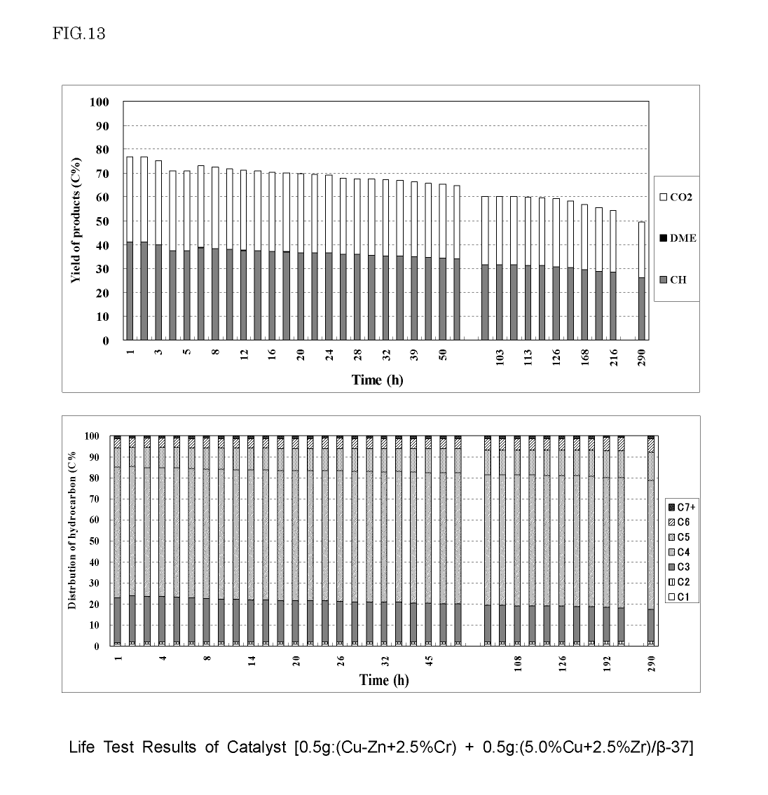

FIG. 13 shows the conversion of carbon monoxide into hydrocarbon (CH), the conversion of carbon monoxide into carbon dioxide by shift reaction, and the composition of the produced hydrocarbon over time. The catalyst wherein Cr was supported on the Cu—Zn-based methanol synthesis catalyst did not exhibit a remarkable effect and the catalytic activity rapidly decreased.

### Example 13

### Preparation of Catalyst

A catalyst was prepared in the same way as Example 11, except that a Cu—Zn-based methanol synthesis catalyst [hereinafter, also referred to as “Cu—Zn+2.5% Zr” in which 2.5 wt % of Zr was supported on a self-made Cu—Zn-based methanol synthesis catalyst (Cu—Zn—Cr composite oxide) by an impregnation method was used as a methanol synthesis catalyst component.

The Cu—Zn-based methanol synthesis catalyst [Cu—Zn+2.5% Zr] was prepared as follows.

First, 0.147 g of ZrO(NO₃)₂.xH₂O was dissolved in 2.5 mL of ion-exchanged water to prepare a Zr-containing solution (concentration: 2.0 wt %). And then, 2 g of Cu—Zn-based methanol synthesis catalyst (Cu—Zn—Cr composite oxide) was added to the resulting Zr-containing solution, and impregnated with the Zr-containing solution for 3 hours. The resulting Cu—Zn-based methanol synthesis catalyst impregnated with the Zr-containing solution was dried at 120° C. for 10 hours, and then calcined at 500° C. for 4 hours. Subsequently, the Zr-supported Cu—Zn-based methanol synthesis catalyst was mechanically pulverized, and molded by a tablet-compression and sized to give a Zr-supported Cu—Zn-based methanol synthesis catalyst having an average particle size of 0.35 to 0.7 mm.

(Production of LPG)

Using the prepared catalyst [(Cu—Zn+2.5% Zr)+(5.0% Cu+2.5% Zr)/β-37], the LPG production reaction and gas chromatographic analysis of the product were carried out in the same way as Example 11.

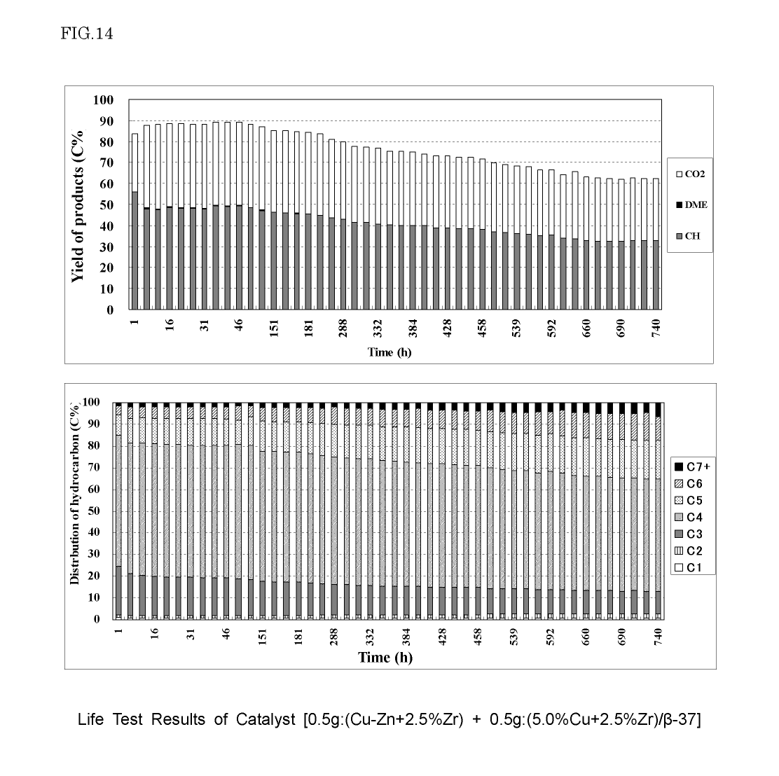

FIG. 14 shows the conversion of carbon monoxide into hydrocarbon (CH), the conversion of carbon monoxide into carbon dioxide by shift reaction, and the composition of the produced hydrocarbon over time. The catalyst wherein Zr was supported on the Cu—Zn-based methanol synthesis catalyst exhibited much higher stability. The catalyst exhibited higher catalytic activity and higher selectivity for LPG, and much less deterioration over time.

The results [the conversion of carbon monoxide into hydrocarbon, the conversion of carbon monoxide into carbon dioxide by shift reaction, and the composition of the produced hydrocarbon] at a reaction time of 50 hours for mixed catalysts of Cu—Zn-based methanol synthesis catalysts and Cu-supported β-zeolites (SiO₂/Al₂O₃ ratio: 37) [Examples 7 to 13] are shown in Table 1.

In all Examples, the LPG production reaction produces substantially no DME.

TABLE 1
Reaction properties of mixed catalysts of Cu—Zn-based methanol synthesis
catalysts and Cu-supported β-zeolites (After 50 hours)

| | | Example 7 | Example 8 | Example 9 |
|---|---|---|---|---|
| Catalyst | Methanol synthesis catalyst | Cu—Zn | Cu—Zn | Cu—Zn |
| | component | | | |
| | Zeolite catalyst component | 5%Cu/β-37 | 10%Cu/β-37 | 2%Cu/β-37 |
| | | | (After 32 hours) | (After 15 hours) |

| | | | |
|---|---|---|---|
| CO conversion (%) | 87.8 | 89.4 | 83 |
| Hydrocarbon yield (C %) | 48.24 | 39.98 | 43.92 |
| DME yield (C %) | 0.1 | 0.03 | 0.02 |
| CO₂ yield (C %) | 39.53 | 49.44 | 39.069 |
| Product (hydrocarbon) composition (%) | | | |
| C1 (methane) | 0.6 | 1.2 | 0.6 |
| C2 (ethane) | 1.4 | 3.5 | 2.6 |
| C3 (propane) | 13.8 | 17.6 | 12.7 |
| C4 (butane) | 58.1 | 56.7 | 58 |
| C5 (pentane) | 15.5 | 14.5 | 17.1 |
| C6+ (hexane etc.) | 10.7 | 6.5 | 9 |
| LPG (C3 + C4) | 71.9 | 74.4 | 70.7 |

| | | Example 10 | Example 11 | Example 12 | Example 13 |
|---|---|---|---|---|---|
| Catalyst | Methanol synthesis catalyst | Cu—Zn | Cu—Zn | Cu—Zn + 2.5%Cr | Cu—Zn + 2.5%Zr |
| | component | | | | |
| | Zeolite catalyst component | (5%Cu + 2.5%Zn)/ | (5%Cu + 2.5%Zr)/ | (5%Cu + 2.5%Zr)/ | (5%Cu + 2.5%Zr)/ |
| | | β-37 | β-37 | β-37 | β-37 |

| | | | | |
|---|---|---|---|---|
| CO conversion (%) | 80.0 | 85.4 | 65.3 | 88.7 |
| Hydrocarbon yield (C %) | 43.11 | 46.28 | 34.35 | 49.18 |
| DME yield (C %) | 0.11 | 0.02 | 0.04 | 0.35 |
| CO₂ yield (C %) | 36.92 | 39.14 | 30.91 | 39.51 |
| Product (hydrocarbon) composition (%) | | | | |
| C1 (methane) | 0.7 | 0.8 | 1.0 | 0.8 |
| C2 (ethane) | 3.5 | 2.7 | 1.0 | 1.0 |
| C3 (propane) | 12.3 | 14.3 | 18.4 | 17.4 |
| C4 (butane) | 52.3 | 56.6 | 62.1 | 12.3 |
| C5 (pentane) | 17.4 | 16.4 | 11.3 | 12.3 |
| C6+ (hexane etc.) | 13.7 | 9.3 | 6.2 | 7.5 |
| LPG (C3 + C4) | 64.6 | 70.9 | 80.5 | 78.4 |

### INDUSTRIAL APPLICABILITY

As described above, a catalyst for producing a liquefied petroleum gas according to the present invention has a longer catalyst life with less deterioration over time, and enables the production of a hydrocarbon containing propane or butane as a main component, i.e. a liquefied petroleum gas (LPG), by reacting carbon monoxide and hydrogen, with high activity, high selectivity and high yield. Therefore, when using the catalyst according to the present invention, LPG may be stably produced from a carbon-containing starting material such as a natural gas or a synthesis gas for a long period with high activity, high selectivity and high yield. Moreover, the catalyst according to the present invention does not comprise high-priced Pd, and therefore the catalyst is more inexpensive than conventional catalysts.

1. A catalyst for producing a liquefied petroleum gas, which is used for producing a liquefied petroleum gas containing propane or butane as a main component by reacting carbon monoxide and hydrogen, comprising
A Cu—Zn-based methanol synthesis catalyst; and
A Cu-supported β-zeolite in which at least Cu is supported on a β-zeolite.

2. The catalyst according to claim 1, wherein a ratio (by weight) of the Cu—Zn-based methanol synthesis catalyst to the Cu-supported β-zeolite [(Cu—Zn-based methanol synthesis catalyst)/(Cu-supported β-zeolite)] is 0.1 to 5.

3. The catalyst according to claim 1, wherein the Cu—Zn-based methanol synthesis catalyst is a composite oxide composed mainly of copper oxide and zinc oxide, or a composite oxide composed mainly of copper oxide and zinc and having at least one metal supported thereon.

4. The catalyst according to claim 3, wherein the composite oxide contains copper oxide and zinc oxide and optionally aluminum oxide and/or chromium oxide in the ratio (by weight) of (copper oxide):(zinc oxide):(aluminum oxide):(chromium oxide)=100:(10 to 70):(0 to 60):(0 to 50).

5. The catalyst according to claim 3, wherein the Cu—Zn-based methanol synthesis catalyst has Zr supported on the composite oxide.

6. The catalyst according to claim 5, wherein the amount of Zr in the Cu—Zn-based methanol synthesis catalyst is 0.5 wt % to 8 wt %.

7. The catalyst according to claim 1, wherein the β-zeolite as a support for the Cu-supported β-zeolite has a SiO2/Al2O3 ratio of 10 to 150.

8. The catalyst according to claim 1, wherein the amount of Cu in the Cu-supported β-zeolite is 0.1 wt % to 15 wt %.

9. The catalyst according to claim 1, wherein the Cu-supported β-zeolite has Cu and Zr supported on the β-zeolite.

10. The catalyst according to claim 9, wherein the amount of Zr in the Cu-supported β-zeolite is 0.1 wt % to 5 wt %.

11. The catalyst according to claim 1, wherein the Cu—Zn-based methanol synthesis catalyst is a composite oxide composed mainly of copper oxide and zinc oxide on which Zr is supported in an amount of 0.5 wt % to 8 wt %; and the Cu-supported β-zeolite is a β-zeolite with a SiO₂/Al₂O₃ ratio of 10 to 150 on which Cu is supported in an amount of 0.1 wt % to 15 wt % and Zr is supported in an amount of 0.1 wt % to 5 wt %.

12. A process for producing a liquefied petroleum gas, comprising:
reacting carbon monoxide and hydrogen in the presence of the catalyst according to claim 1, thereby producing a liquefied petroleum gas containing propane or butane as a main component.

13. The process according to claim 12, wherein carbon monoxide and hydrogen are reacted at a reaction temperature of 260° C. to 325° C.; a reaction pressure of 1.6 MPa to 4.5 MPa; and a contact time between a starting gas, which contains carbon monoxide and hydrogen, and the catalyst [W/F; ratio of the weight of the catalyst (W; g) to the total flow rate of the starting gas (F; mol/h)] of 2 g·h/mol to 20 g·h/mol.

14. A process for producing a liquefied petroleum gas, comprising:
feeding a synthesis gas to a catalyst layer comprising the catalyst according to Claim 1, thereby producing a liquefied petroleum gas containing propane or butane as a main component.

15. A process for producing a liquefied petroleum gas, comprising:
producing a synthesis gas from a carbon-containing starting material and at least one selected from the group consisting of H₂O, O₂ and CO₂; and
feeding the synthesis gas to a catalyst layer comprising the catalyst according to claim 1, thereby producing a liquefied petroleum gas containing propane or butane as a main component.
